(19) United States
(12) Patent Application Publication
(10) Pub. No.: US 2025/0344955 A1
(43) Pub. Date: Nov. 13, 2025

(54) Predicting and Managing Congestive Heart Failure Based on Blood Pressure Measurements Received from an Implanted Device

(71) Applicant: VECTORIOUS MEDICAL TECHNOLOGIES LTD., Tel-Aviv (IL)

(72) Inventors: Maoz Handelman, Hod Hasharon (IL); Tom Marianer, Tel Aviv (IL); Matan Hershko, Tel Aviv (IL); Alon Rabinowicz, Kfar Saba (IL); Eyal Orion, Ramat Gan (IL)

(21) Appl. No.: 18/869,307

(22) PCT Filed: Jun. 7, 2023
(86) PCT No.: PCT/IB2023/055882

§ 371 Date: Nov. 26, 2024

Related U.S. Application Data
(60) Provisional application No. 63/350,439, filed on Jun. 9, 2022.

(51) Int. Cl. A61B 5/021; A61B 5/00

(52) U.S. Cl. CPC A61B 5/021; A61B 5/7246; A61B 5/725; A61B 5/7275; A61B 5/6847; A61B 2560/0228

(57) ABSTRACT

A method includes receiving a plurality of measurements of blood pressure acquired in a heart (28) of a patient (30). A periodic waveform of the blood pressure is derived from the measurements, and one or more parameters (Vpeak 45, Apeak 43, RVL, RAL, mean LAP, rise rate, fall rate, rise time, fall time) of one or more components (AW, VW, RP) of the periodic waveform, respectively, are estimated. Occurrence of a cardiac condition in the patient is predicted based on the estimated one or more parameters.

### CROSS-REFERENCE TO RELATED APPLICATIONS

This application claims the benefit of U.S. Provisional Patent Application 63/350,439, filed Jun. 9, 2022, whose disclosure is incorporated herein by reference.

### FIELD OF THE INVENTION

The present invention relates generally to medical devices, and particularly to methods and systems for managing congestive heart failure in a patient based on blood pressure measurements obtained using an implanted device.

### BACKGROUND OF THE INVENTION

Various techniques for: (i) acquiring signals indicative of blood pressure measurements in the heart of a patient, and (ii) analyzing the signals, have been published.

For example, U.S. Pat. No. 10,687,716 describes a method comprising using a pressure sensor for sensing the ambient pressure in a living organ in which the ambient pressure varies as a function of time. The pressure sensor has a capacitance that varies in response to the ambient pressure, so as to produce a time-varying waveform.

U.S. Pat. No. 11,206,988 describes an apparatus that includes an antenna configured to, by drawing energy from a magnetic field, provide a main supply voltage. The apparatus further comprises operational circuitry configured to operate only if a derived supply voltage, derived from the main supply voltage and supplied to the operational circuitry, is greater than a threshold value.

U.S. Pat. No. 11,642,084 describes an apparatus comprising a magnetic-field transducer, and circuitry. The magnetic-field transducer is configured to be coupled externally to a body of a patient. The circuitry is configured to generate and apply to the magnetic-field transducer a time-varying signal, so as to generate a time-varying magnetic field outside the body of the patient, for supplying electrical energy by inductive coupling to an electronic device that is positioned inside the body, to estimate an intensity of the magnetic field that reaches the electronic device, and to assess fluid retention in an organ of the patient based on the estimated intensity of the magnetic field.

### SUMMARY OF THE INVENTION

An embodiment of the present invention that is described herein provides a method including receiving a plurality of measurements of blood pressure acquired in the heart of a patient. A periodic waveform of the blood pressure is derived from the measurements, and one or more parameters of one or more components of the periodic waveform, respectively, are estimated. An occurrence of a cardiac condition in the patient is predicted based on the estimated one or more parameters.

In some embodiments, receiving the measurements include receiving multiple ones of the measurements of the blood pressure per cardiac cycle. In other embodiments, the blood pressure measurements include Left Atrial Pressure (LAP) measurements acquired by a cardiac implant. In yet other embodiments, the one or more components of the periodic waveform include at least one of: (i) a ventricle wave (VW) generated in response to a passive filling of an atrium of the heart with oxygenated blood, and (ii) an atrial wave (AW) generated in response to an active contraction of the atrium.

In some embodiments, estimating the one or more parameters includes estimating at least one of: (i) a first peak pressure of the VW (Vpeak), and (ii) a second peak pressure of the AW (Apeak). In other embodiments, the blood pressure measurements include Left Atrial Pressure (LAP), and estimating the one or more parameters include calculating a mean LAP, which is an average of the measurements of the LAP in the periodic waveform. In yet other embodiments, the method includes detecting in the periodic waveform: (i) a first local minimum LAP at a first side of the Vpeak, and (ii) a second local minimum LAP at a second side of the Vpeak, opposite the first side.

In some embodiments, estimating the one or more parameters include estimating a rise rate, by (i) calculating a first LAP difference between the first local minimum LAP and the Vpeak, and (ii) dividing the first LAP difference by a rise time parameter, which is a first time interval between the first local minimum LAP and the Vpeak. In other embodiments, estimating the one or more parameters include estimating a fall rate, by (i) calculating a second LAP difference between the Vpeak and the second local minimum LAP, and (ii) dividing the second LAP difference by a fall time parameter, which is a second time interval between the Vpeak and the second local minimum LAP. In yet other embodiments, estimating the one or more parameters include estimating at least one of: (i) a relative ventricle LAP (RVL), by subtracting the mean LAP from the Vpeak, and (ii) a relative atrial LAP (RAL), by subtracting the mean LAP from the Apeak.

In some embodiments, when (i) the mean LAP exhibits a trend as a function of time and (ii) at least one of the RVL and RAL does not exhibit the trend, predicting occurrence of the cardiac condition is based on at least one of the RVL and RAL. In other embodiments, the method includes calibrating the acquisition of the measurements of the blood pressure responsively to a difference between the mean LAP and at least one of RVL and RAL. In yet other embodiments, when both (i) the mean LAP and (ii) at least one of the RVL and RAL exhibit a trend as a function of time, predicting occurrence of the cardiac condition is based on the trend of one or both of: (a) the mean LAP, and (b) at least one of the RVL and RAL.

In some embodiments, the method includes plotting: (i) a first graph of a first moving average of the mean LAP as the function of time, and (ii) one or more second graphs of second moving averages of one or both of the RVL and the RAL as the function of time, respectively. In other embodiments, the method includes calculating a correlation between the mean LAP and at least one of RVL and RAL, and determining a threshold indicative of an occurrence of a heart failure exacerbation (HFE), and predicting occurrence of the cardiac condition includes predicting the HFE when the calculated correlation exceeds the threshold. In yet other embodiments, the measurements exhibit a trend as a function of time, and estimating the parameter includes canceling at least part of the trend.

In some embodiments, estimating the one or more parameters includes: (a) identifying in the periodic waveform: (i) one or more first peaks indicative of one or more maximum values of the blood pressure within one or more time intervals of the periodic waveform, respectively, (ii) one or more second peaks indicative of one or more minimum values of the blood pressure within the one or more time intervals, respectively, and (b) estimating a pressure difference between each pair of the first and second peaks within each of the time intervals. In other embodiments, the method includes predicting the occurrence of the cardiac condition based on the one or more estimated pressure differences. In yet other embodiments, estimating the one or more parameters includes: (a) calculating a mean blood pressure, which is an average of the measurements of the blood pressure in the periodic waveform, and (b) estimating a pressure amplitude by subtracting the mean blood pressure from at least one of the first and second peaks, and including predicting the occurrence of the cardiac condition based on the estimated pressure amplitude.

In some embodiments, the method includes determining, for at least a given parameter among the one or more parameters, at least a first range of first values and a second range of second values different from the first values, and predicting the occurrence of the cardiac condition includes comparing between: (a) a given value of the given parameter, and (b) the first and second ranges of the first and second values. In other embodiments, the method includes determining at least one of: (i) a first treatment to the patient, in case the given value is within the first range, (ii) a second treatment to the patient, in case the given value is within the second range, and (iii) a third treatment to the patient, in case the given value is out of the first and second ranges.

In some embodiments, the method includes (i) receiving a plurality of additional measurements of another blood pressure acquired in another heart of an additional patient; (ii) deriving, from the additional measurements, an additional periodic waveform of the another blood pressure, and estimating the one or more parameters of the one or more components identified in the additional periodic waveform, respectively; and (iii) predicting the occurrence of the cardiac condition in the additional patient based on the estimated one or more parameters. In other embodiments, the method includes setting (i) a first threshold for predicting the occurrence of the cardiac condition in the patient, and (ii) a second threshold, different from the first threshold, for predicting the occurrence of the cardiac condition in the additional patient.

There is additionally provided, in accordance with an embodiment of the present invention, a system including an interface and a processor. The interface is configured to receive a plurality of measurements of blood pressure acquired in the heart of a patient. The processor is configured to: (i) derive, from the measurements, a periodic waveform of the blood pressure, and estimate one or more parameters of one or more components of the periodic waveform, respectively, and (ii) predict occurrence of a cardiac condition in the patient based on the estimated one or more parameters.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for combined assessment of body fluid retention and Left-Atrial (LA) blood pressure (LAP), in accordance with an embodiment of the present invention;

FIG. 2 is a schematic, pictorial illustration of a graph that depicts measurements of LAP over time in the LA of a patient, in accordance with an embodiment of the present invention;

FIGS. 3A and 3B, are schematic presentations of graphs depicting moving averages of parameters calculated based on waveforms (WFs) of the LAP measured in the patient over a period of time using the system of FIG. 1, in accordance with embodiments of the present invention;

FIG. 4A is a schematic presentation of a graph depicting moving average of parameters calculated based on another WF of LAP measured in another patient over a period of time using the system of FIG. 1, in accordance with embodiments of the present invention;

FIGS. 4B and 4C are schematic presentations of graphs depicting first and second levels of correlation between the parameters of FIG. 4A in first- and second-time intervals, respectively, which are located within the period of time of FIG. 4A, in accordance with embodiments of the present invention;

FIGS. 5 and 6 are schematic presentations of multiple points annotated over a graph depicting a mean LAP calculated based on LAP measurements carried out over several months in a patient using the system of FIG. 1, in accordance with embodiments of the present invention;

FIG. 7A is a schematic presentation of another graph depicting parameters calculated based on LAP measured in the hearts of several patients using the system of FIG. 1, in accordance with an embodiment of the present invention;

FIG. 7B is a schematic presentation of an additional graph depicting parameters calculated based on LAP measured in the hearts of other patients using the system of FIG. 1, in accordance with an embodiment of the present invention;

FIG. 8 is a schematic presentation of another graph depicting techniques for estimating peaks of blood pressure based on LAP measurements acquired over a predefined time interval using the system of FIG. 1, in accordance with an embodiment of the present invention;

FIG. 9 is a flow chart that schematically illustrates a method for estimating peaks and parameters based on LAP measurements acquired over a predefined time interval using the system of FIG. 1, in accordance with an embodiment of the present invention;

FIGS. 10 and 11 are flow charts that schematically illustrate methods for treating congestive heart failure (CHF) in the patient heart based on the parameters calculated in FIG. 9 above, in accordance with embodiments of the present invention;

FIG. 12 is a schematic presentation of another graph of simulated signals indicative of a WF of LAP measurements, and calculation of several parameters based on the simulated WF, in accordance with an embodiment of the present invention;

FIG. 13 is a flow chart that schematically illustrates a method for estimating (i) rise rate and/or rise time, and (ii) fall rate and/or fall time between adjacent maximum and minimum peaks of LAP measurements, in accordance with an embodiment of the present invention;

FIG. 14 is a flow chart that schematically illustrates a method for treating CHF in the patient heart based on at least one parameter among the parameters of: (i) peak-to-peak (P2P), (ii) LAP amplitude, (iii) rise rate and/or rise time, and (iv) fall rate and/or fall time that are calculated in FIG. 13 above, in accordance with an embodiment of the present invention;

FIG. 15 is a flow chart that schematically illustrates a method for estimating change rate in one or more peaks of the LAP and mean LAP, and for assessing whether the mean LAP change rate is applicable for treating CHF, in accordance with an embodiment of the present invention;

FIG. 16 is a flow chart that schematically illustrates a method for treating CHF in the patient heart based on the calculated change rate in at least one of the one or more peaks and the mean LAP described in FIG. 15, in accordance with an embodiment of the present invention;

FIG. 17 is a flow chart that schematically illustrates a method for estimating the heart rate of a patient based on the LAP measurements received from an implanted device of the system of FIG. 1, in accordance with an embodiment of the present invention; and

FIG. 18 is a flow chart that schematically illustrates a method for estimating the respiration rate of a patient based on the LAP measurements received from the implanted device of the system of FIG. 1, in accordance with an embodiment of the present invention.

### DETAILED DESCRIPTION OF EMBODIMENTS

### Overview

Several methods of treating chronic heart failure (CHF) in patients, and preventing congestion and recurrent hospitalizations of such patients are known in the art. Emerging treatment paradigm that rely on hemodynamic monitoring the mean pressure of pulmonary artery of the patient has been shown to reduce recurrent hospitalization of CHF patients. This method examine the mean pressure, i.e., an average of a continuously pressure sampled over a few seconds, and responding to different values of the mean pressure by adjusting medication in order to adjust fluid volume and pressure back to the optimal status. Left Atrial Pressure (LAP) is considered the gold standard indicator for congestion and volume overload. In the acute clinical setting, the mean LAP, assessed by a Swan Ganz catheter, serves today as the gold standard parameter for hemodynamic management of acute heart failure. LAP measurements for CHF patients in the ambulatory setting, which are within the context of the present disclosure, is novel treatment paradigm which will enable a more accurate and reliable management of CHF patients. In the context of the present disclosure, the terms hemodynamic management and grammatical variations thereof, refer to assessment of the patient status, determination of a suitable treatment, and monitoring the patient status responsively to the treatment.

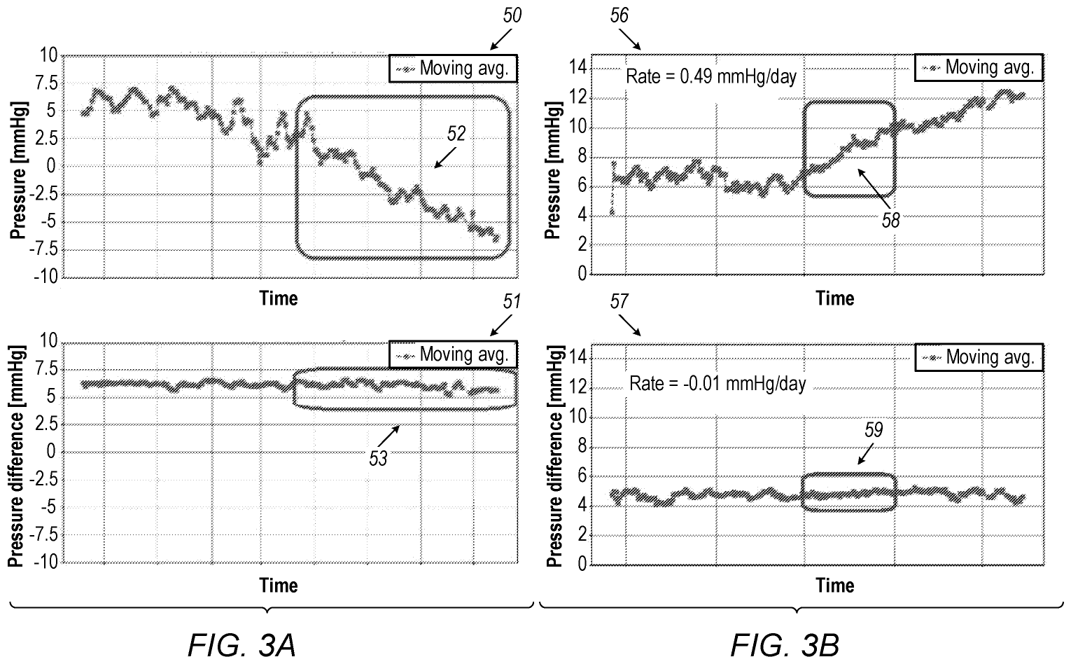

In some cases, the mean pressure in general, or LAP in particular, may not provide the healthcare provider (e.g., a cardiologist) with accurate and reliable information for: (i) assessing the current status, and determining a suitable treatment required for the patient in question, and (ii) monitoring the patient status after receiving the treatment. Examples of such cases are depicted in detail, for example, in FIGS. 3A, 3B, 4A, 4B, and 7B below.

Embodiments of the present invention that are described herein below provide techniques for broadening the scope of hemodynamic management, and improving the quality and reliability thereof. More specifically, the disclosed techniques add components to the hemodynamic management and help the healthcare provider in: (i) assessing the present cardiac condition of a patient in question, (ii) predicting occurrence of a congestive heart failure (CHF) exacerbation. (iii) determining and managing a suitable (and typically proactive) treatment, (iv) monitoring the status of the patient heart responsively to the treatment, and (v) controlling and improving the accuracy of the LAP measurements.

In some embodiments, a system for delivering the above components of hemodynamic management comprises an interface and a processor. The interface is configured to receive from a sensor, implemented in the heart and described herein, a plurality of the LAP measurements described above.

In some embodiments, the LAP is measured using an implanted device (also referred to herein as an implant, for brevity) having a pressure sensor for measuring the LAP. The implant is configured to generate signals indicative of the LAP measurements, and to send the signals to an external device configured to provide the implant with electrical power and instructions. In the present example, the signals received from the implant are transferred to a cloud-based server (and/or to any other suitable computer), which performs the calculations of the continuous pressure measured by the implant and generates a periodic waveform (WF) described in detail below. It is noted that embodiments of the signal analysis and treatment methods described herein, are applicable to any suitable LAP measurements received from any suitable type of pressure sensor capable of continuously measuring the blood pressure in the left atrium of the patient's heart.

The WF of each heartbeat cycle that is sampled using the pressure sensor implanted in the left atrium, typically contains two adjacent waves followed by a resting period. The first wave is generated responsively to the active contraction of the atrium, and is referred to herein as an A wave (AW). The second wave is generated responsively to the passive filling of the atrium with oxygenated blood from the lungs and the contraction of the ventricle, and is referred to herein as the V wave (VW).

In some embodiments, the processor is configured to derive, from the LAP measurements, the periodic WF of the LAP. In the context of the present disclosure and in the claims, the adjacent waves and the resting periods are also referred to herein as components of the periodic waveform derived by the processor based on the LAP measurements. It is noted that the periodicity of the waveform corresponds to the periodicity of the heartbeats.

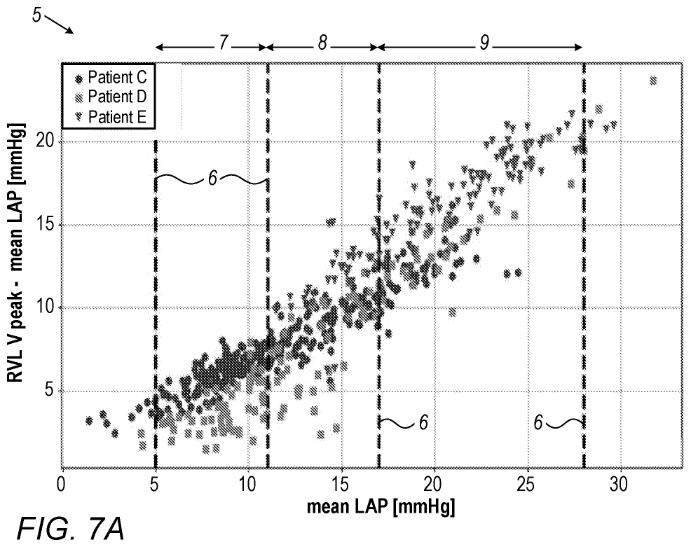

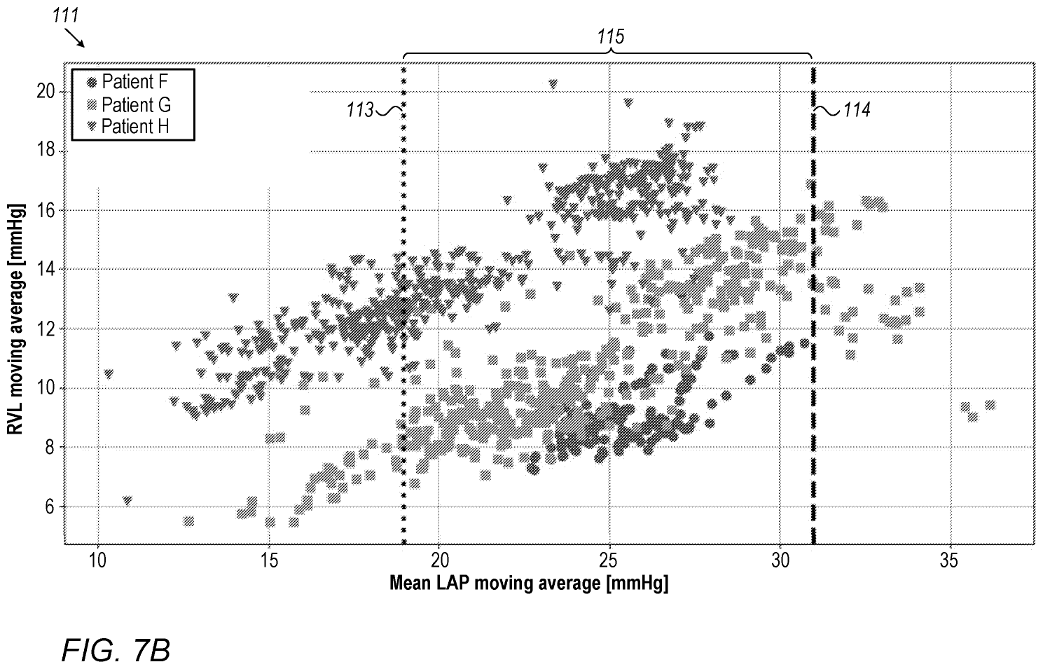

In some embodiments, analysis of the waveform provides users of the system with insights to the biomechanical condition (i.e., compliance) and comorbidities (i.e., valvular disease) of the heart of each patient, and thereby, enables personalization of the monitoring and treatment to each individual patient, as will be described in detail in FIGS. 7A and 7B below.

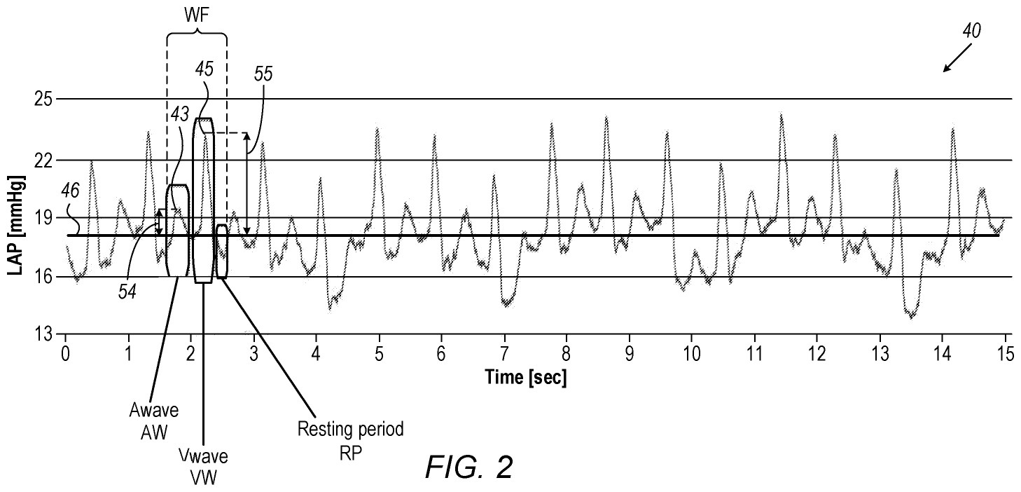

In some embodiments, the processor is further configured to estimate one or more parameters of one or more of the components of the periodic waveform, respectively. The estimated parameters are described in detail below, for example, in FIGS. 2, 4A-6, 7A-8 and 12.

In some embodiments, based on (i) the estimated one or more parameters, and optionally, (ii) additional parameters calculated using two or more of the estimated parameters, the processor is configured to provide the healthcare provider with: (a) an accurate assessment of the present cardiac condition of a patient, and more important, (b) a prediction and early warning for occurrence of a deterioration in the cardiac and overall condition of the patient. Moreover, the processor is configured to provide the healthcare provider with techniques for (i) proactively treating the patient being monitored by the system, before the development of the CHF exacerbation, and thereby to reduce or eliminate the occurrence of hospitalization, and (ii) Improve the accuracy of the LAP measurements in reflecting the congestion status of the patient, e.g., by applying the disclosed techniques for detecting and suppressing undesired drifting or other inaccuracies in the LAP measurements, which are related to the measurement and not to the actual congestion status of the patient.

As such, the disclosed techniques are used for analyzing the WF, and specifically V waves and A waves of the WF, in addition to the mean LAP, so as to provide users of the system with significant clinical value by optimizing and personalizing the hemodynamic management in patients.

### System Description

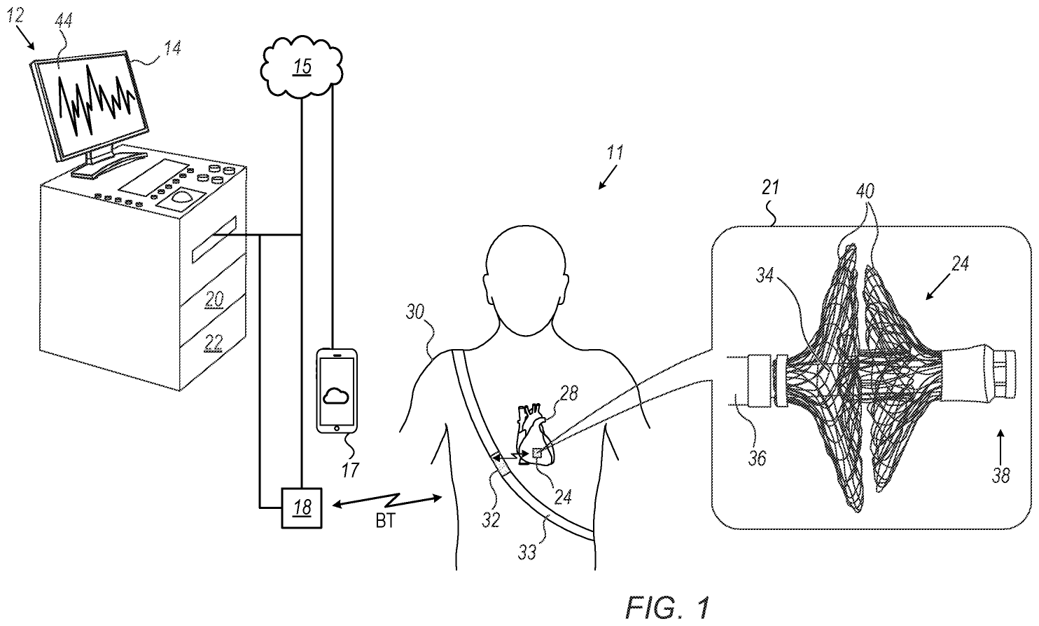

FIG. 1 is a schematic, pictorial illustration of a system 11 for combined assessment of body fluid retention and Left-Atrial (LA) blood pressure, in accordance with an embodiment of the present invention. System 11 comprises an implanted device, also referred to herein as an implant 24, which is implanted at a desired location in a heart 28 of a patient 30, and is used for measuring the ambient blood pressure in its vicinity. In an example embodiment, implant 24 is implanted across the interatrial septum of heart 28, and is configured to measure the blood pressure in the Left Atrium (LA).

System 11 further comprises an external unit 32, which is configured to communicate with implant 24 and to provide electrical power to the implant's circuitry. In the present example, external unit 32 is fitted on a belt that is worn by the patient. The belt also comprises an antenna coil 33 of the external unit that surrounds the patient's thorax. In the present example the belt is worn diagonally over the neck and one shoulder of the patient. Alternatively, however, any other suitable configuration can be used.

Implant 24 typically does not comprise an internal power source. The internal circuitry of the implant is powered by energy that is provided by external unit 32 using inductive coupling. Typically, the external unit generates an Alternating Current (AC) magnetic field via antenna coil 33. This magnetic field induces an AC voltage across an antenna of the implant, and this voltage is then rectified and used for powering the implant circuitry. At the same time, the implant sends data (e.g., measurement results of ambient blood pressure) by modulating the load impedance of its antenna, modulation that is sensed by the external unit.

Reference is now made to an inset 21 showing the mechanical structure of implant 24. In this example embodiment, implant 24 comprises an elongated tube 34 that comprises the electronic circuitry of the implant. Tube 34 is inserted into the interatrial septum. A “septum gripper” 40, comprising a collapsible and extensible mesh, is used for fixating tube 34 to the septum. An antenna coil 36 and a pressure sensor 38 are fitted on opposite sides of tube 34. Implant 24 is implanted such that pressure sensor 38 is positioned in the left atrium and antenna 36 is in the right atrium.

Implants of this sort are addressed in greater detail in U.S. Patent Application Publication 2018/0110468, entitled “Heart Implant with Septum Gripper” and in U.S. Patent Application Publication 2018/0098772, entitled “Deploying and Fixating an Implant Across an Organ Wall,” which are assigned to the assignee of the present patent application and whose disclosures are incorporated herein by reference.

Further aspects of blood pressure measurement using such implants, and of interaction between implants and external units using magnetic-field inductive coupling, are addressed, for example, in U.S. Patent Application Publication 2015/0282720, entitled “Drift Compensation for Implanted Capacitance-Based Pressure,” in U.S. Pat. No. 10,105,103, entitled “Remotely Powered Sensory Implant,” in U.S. Patent Application Publication 2019/0008401, entitled “Power-Efficient Pressure-Sensor Implant,” and in U.S. Pat. No. 10,205,488, entitled “Low-Power High-Accuracy Clock Harvesting in Inductive Coupling Systems.” All these patents and patent applications are assigned to the assignee of the present patent application and their disclosures are incorporated herein by reference.

In some embodiments, in addition to external unit 32 and antenna coil 33 the belt is electrically connected to a power source (not shown), such as a rechargeable battery. The belt may be worn by patient 30 out of the hospital (e.g., at home) or at the hospital when patient 30 is being hospitalized, e.g., in cases described below. It is noted that the (i) blood pressure measurements, and (ii) the communication between external unit 32 and implant 24, are carried out during one or more daily time intervals (e.g., each time interval has a duration between a few minutes and one hour), and the battery is being charged by the electrical grid not during these time intervals in order to prevent noise from the electrical grid to interfere with the blood pressure measurements.

In some embodiments, external unit 32 comprises a wireless communication device configured to transmit signals comprising raw data indicative of the blood pressure measurements. System 11 comprises a cloud gateway device 18 configured to exchange signals with the wireless communication device of external unit 32. In the present example, the signals are exchanged using Bluetooth™ (BT) or using any other suitable communication protocol and devices. Cloud gateway device 18 is configured to transmit the signals to a cloud computing system, referred to herein as a cloud 15, which is configured to analyze the signals, and to display analyzed data described in detail below.

Additionally, or alternatively, gateway device 18 is configured to transmit the signals to a computer 12 of system 11 used by healthcare provider (not shown). In other embodiments, cloud gateway device 18 may be integrated in computer 12 or in any other suitable device or computing system.

In the present example, the analyzed data is transmitted from cloud 15 to computer 12, and at least a portion of the analyzed data is transmitted to a patient self-management web-based application installed on a mobile device 17 (e.g., a smartphone) of patient 30.

In some embodiments, computer 12 comprises a processor 22, in the context of the present disclosure and in the claims, the term “processor” refers to one or more of the following devices: (i) any suitable type of a central processing unit (CPU) such as but not limited to a general-purpose processor, (ii) a graphical processing unit (GPU), (iii) a tensor processing unit (TPU), (iv) a digital signal processor (DSP), and (v) any other suitable type of an application-specific integrated circuit (ASIC). At least one of, and typically all the above types of processing units may have suitable front end and interface circuits configured for interfacing and exchanging signals with (a) several modules and stations of system 11, and (b) entities external to system 11.

Additionally, or alternatively, computer 12 comprises an interface 20, which is configured to exchange data between processor 22 and other entities of system 11 and/or external to system 11, such as cloud 15.

In some embodiments, processor 22 and the electronic circuitry of the implant may be programmed in software to carry out the functions that are used by system 11, and store data for the software in a memory (not shown). The software may be downloaded to processor 22 and to the electronic circuitry of the implant in electronic form, over a network, for example, or it may be provided on non-transitory tangible media, such as optical, magnetic, or electronic memory media.

In some embodiments, computer 12 comprises a display device, referred to herein as a display 14, which is configured to display to the healthcare provider (e.g., a cardiologist) an image 44, such as a graph and/or data of the analyzed blood pressure measurements received from (i) processor 22, and/or (ii) cloud 15.

FIG. 2 is a schematic, pictorial illustration of a graph 40 that depicts measurements of blood pressure over time in the LA of patient 30, in accordance with an embodiment of the present invention.

In the context of the present disclosure and in the claims, the term left atrial pressure (LAP) refers to the blood pressure, which is measured using system 11 over a selected time interval, in the left atrium of patient 30 or any other patient, as will be described in more detail below.

Moreover, in the context of the present disclosure and in the claims, the terms “processor,” and “processor 22” are used interchangeably and refer to any suitable processing unit implemented in cloud 15 and/or in computer 12, which is configured to carry out at least one of the following activities: deriving periodic waveforms, analyzing the waveforms and estimating parameters of components of the waveforms, and predicting occurrence of a cardiac condition, such as congestive heart failure (CHF) also referred to herein as progressive irreversible disease, as will be described in detail below.

In the present example, the LAP is measured during a time interval of about 15 seconds, and has mmHg units. In some embodiments, processor 22 is configured to derive from graph 40 a plurality of periodic waveforms (WFs) corresponding to a plurality of heartbeat cycles of heart 28 of patient 30. The frequency of the heart beats is about 1 Hz, and therefore, graph 40 comprises about 15 WFs. Note that the WFs are riding the respiratory (respiration) wave of patient 30. The respiration wave has a frequency of about 0.2 Hz and a variable amplitude, which is the main reason for the difference in the LAP among the WFs.

Each WF has three components:
(i) An atrial wave (AW) of the LAP is generated in response to the active contraction of the atrium of the heart. A peak pressure of the AW is referred to herein as an Apeak 43,
(ii) A ventricle wave (VW) of the LAP is generated in response to the passive filling of the atrium with oxygenated blood received from the lungs of patient coupled with contraction of the ventricle 30. A peak pressure of the VW is referred to herein as a Vpeak 45, and
(iii) A resting period (RP) of the heart before the AW of the next WF.

In some embodiments, a controller or control circuitry (not shown) of system 11 is configured to control implant 24 to apply any suitable sampling rate of the LAP measurements. The processor (e.g., a processor of cloud 15 and/or processor 22) is configured to calculate an average LAP of all the LAP measurements of graph 40. In the present example, graph 40 has 15 WFs, and the LAP sampling each WF comprises about 100 measurements of the LAP in the heart of patient 30, therefore, graph 40 comprises about 1500 measurements of the LAP. The average LAP is also referred to herein as a mean LAP (ML) 46.

In other embodiments, the aforementioned controller or control circuitry is configured to set any other suitable sampling rate, such as but not limited to about 50 LAP measurements per second. It is noted that presently known mechanical mechanisms in the heart have typical frequencies between almost 0 Hz (e.g., a minor change that occurs every several days or weeks) and about 25 Hz. Thus, the sampling rate of the LAP measurements by implant 24 is typically determined by the mechanical mechanism that the cardiologist wants to explore, and the type of data required for the monitoring and the treatment.

In some embodiments, the processor (e.g., processor 22 or a processor implemented in cloud 15) is configured to calculate one or more parameters that may be used for analyzing the components of graph 40. In an embodiment, processor 22 is configured to calculate a parameter of the VW by subtracting the value of ML 46 from the value of Vpeak 45, and the calculated parameter is referred to herein as a relative V-LAP (RVL) 55. Additionally, or alternatively, processor 22 is configured to calculate a parameter of the AW by subtracting the value of ML 46 from the value of Apeak 43, and the calculated parameter is referred to herein as a relative A-LAP (RAL) 54.

In some embodiments, the parameters of relative LAP, such as RVL 55 and RAL 54, may be used for: (i) monitoring the heart condition of patient 30, (ii) predicting and alerting the development of CHF and/or other sorts of heart failure exacerbations, and (iii) detecting false alarms related to measurements of the LAP rather than to the heart condition and congestion status, as will be described in detail below.

### Predicting a Condition of the Heart by Estimating a Parameter in Periodic WF of the Blood Pressure

FIG. 3A is a schematic presentation of graphs 50 and 51 depicting moving averages of parameters calculated based on the waveform of the LAP measured in patient 30 over a period of nine months using system 11, in accordance with an embodiment of the present invention. In the present example, each WF is generated based on the LAP measured during a time interval of about 15 seconds (or any other suitable time interval), and graphs 50 and 51 are generated using processor 22 and/or any processor of cloud 15, which are also referred to herein as processor, for generalization.

In some embodiments, the moving average is calculated by the processor using LAP measurements acquired by implant 24 over five days. It is noted that the moving average is calculated for smoothing the shape of graphs 50 and 51, so as to identify trends. In other embodiments, any other suitable smoothing technique may be applied to the LAP measurements received from implant 24.

In some embodiments, graph 50 comprises the 5-day moving average of the mean LAP measured in the heart of patient 30. In the present example, a section 52 of graph 50 is trending down.

In the context of the present disclosure, the documents incorporated by reference, and in the claims, the terms trending down, down trending, fall rate, descent, and grammatical variations thereof, are used interchangeably, and refer to a rate of reduction over time in one or more parameters, such as Vpeak, Apeak, mean LAP, RVL and RAL described in FIG. 1 above. Similarly, the terms trending up, uptrend, rise rate, ascent, and grammatical variations thereof, are used interchangeably, and refer to an increased rate over time in one or more of the aforementioned parameters.

In some embodiments, in graph 51 the processor is configured to (i) calculate a relative V-LAP (RVL) by subtracting, in each waveform, the mean LAP from an average of the Vpeaks, and (ii) calculate a 5-day moving average of the RVLs calculated based on the WFs produced within the respective five days. In the present example, the time interval of a section 53 of graph 51 corresponds to that of section 52 of graph 50. The calculated trend of sections 52 is about-0.39 mmHg/day, and the calculated trend of sections 53 is about-0.01 mmHg/day.

In other words, the processor is applying the waveform analysis described above (e.g., calculating a 5-day moving average of the RVL), to detect a drift in the measurements carried out by implant 24, and thereby, to prevent the administration of a wrong treatment to patient 30. In some embodiments, the waveform analysis described above is applicable, mutatis mutandis, for analyzing LAP measurements received from additional types of sensors (other than implant 24), and system 11 has additional techniques for detecting a drift in implant 24. Moreover, in response to detecting the drift, the processor is configured to perform a corrective action, such as a calibration of implant 24, in order to suppress the drift. The corrective action may be carried out automatically (when implant is idle), or after receiving permission to do so from the healthcare provider.

FIG. 3B is a schematic presentation of graphs 56 and 57 depicting moving averages of parameters calculated based on the LAP measured in a patient A (not shown), other than patient 30, over a period of about 4.5 months using system 11, in accordance with an embodiment of the present invention.

In some embodiments, graphs 56 and 57 are generated using the processor and present a 5-day moving average of the LAP measurements, as described in FIG. 3A above. Graph 56 presents the mean value, and graph 57 presents the RVL, which is calculated by subtracting the mean lap from the Vpeak, using the same techniques described in FIG. 3A above. Moreover, a section 58 of graph 56 and a section 59 of graph 57 present the mean LAP and the RVL of the LAP measurements acquired in the heart of the patient A over one month, respectively.

In the present example, the processor calculates in section 58 a slope of about 0.49 mmHg/day, and in a negligible slope of about-0.01 mmHg/day in section 59.

In such embodiments, based on the WF analysis techniques described above, the processor is configured to identify a trending-up drift, which is related to the measurement and is not indicative of congestion or heart failure exacerbation of patient A.

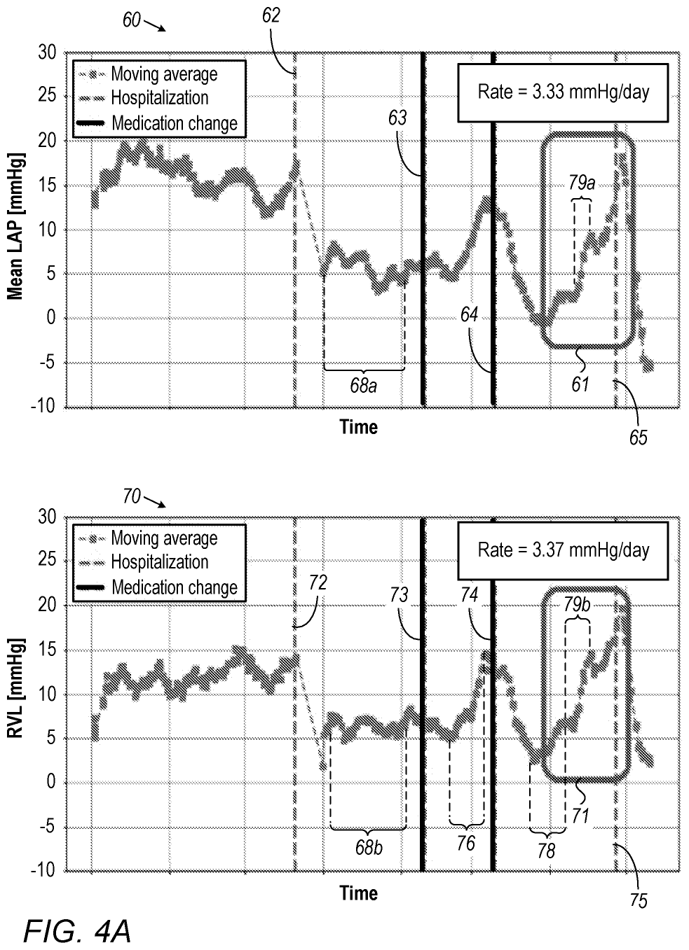

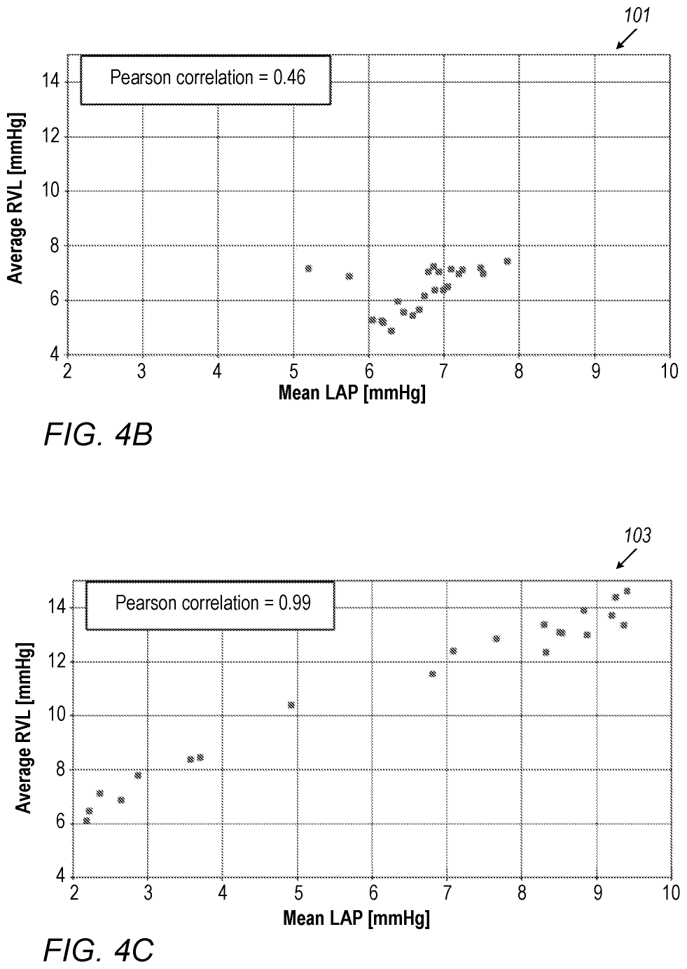

FIG. 4A is a schematic presentation of graphs 60 and 70 depicting moving averages of parameters calculated based on the LAP measured in a patient B (not shown), other than patient 30, over a period of about 7.5 months using system 11, in accordance with an embodiment of the present invention.

In some embodiments, the processor applies the waveform analysis techniques described in FIG. 3A above, to produce graphs 60 and 70 based on the LAP measurements performed by implant 24 in the heart of patient B. In the present example, graph 60 presents a 5-day moving average of the mean value, and graph 70 presents a 5-day moving average of the RVL. Moreover, a section 61 of graph 60, and a section 71 of graph 70 present the mean LAP and the RVL of the LAP measurements acquired in the heart of the patient B over about 5 weeks, respectively.

In principle, the cardiologist could try to assess the cardiac condition of patient B based on the LAP measurement and/or the calculated mean LAP. In some cases, however, this information is not accurate and may be misleading, as shown for example in FIGS. 3A and 3B above. In some embodiments, by combining the WF analysis with the calculated mean LAP, the processor is configured to provide the healthcare provider with an improved accuracy of the cardiac condition and congestion status, and a prediction of possible occurrence of a heart failure exacerbation (HFE). For example, based on the measurements received from implant 24, the cardiologist wants to know whether a trend in the LAP and/or mean LAP is indicative of a CHF build up, and in case it is, what is the rate of deterioration in the cardiac condition resulting in severe HFE. It is noted that in many cases, a slight adjustment of the medication administration (rather than aggressive treatment) is sufficient to overcome an increase in the mean LAP.

In some embodiments, the WF analysis provides the healthcare provider with a layer of information (in addition to that of the LAP and mean LAP), which is based on the biomechanical properties of the heart of each individual patient. For example, the processor is configured to estimate the cardiac condition based on one or more of: (i) an increase in RVL and/or in the rise rate thereof, and (ii) the level of correlation between the RVL and the mean LAP, as will be described in detail below.

In the present example, the processor calculates in section 61 a rise rate of about 3.33 mmHg/day in the mean LAP, and an RVL rise rate of about 3.37 mmHg/day in section 71. It is noted that a simultaneous steep rise rate of both the RVL and the mean LAP is indicative of a rapid CHE in the heart, and therefore, an immediate aggressive treatment is required for stabilizing the cardiac condition of patient B. Thus, the steep slope of the mean LAP in the example of section 61 is indicative of the development of CHF in the patient B.

In such embodiments, based on the techniques described above, the processor is configured to identify a trending-up congestion and blood pressure, which is indicative of a failure exacerbation in the heart of the patient B.

In the present example, (i) dashed lines 62 and 72 of graphs 60 and 70, respectively, and (ii) dashed lines 65 and 75 of graphs 60 and 70, respectively, are indicative of first and second hospitalization events of patient B, respectively. Moreover, (i) dashed lines 63 and 73 of graphs 60 and 70, respectively, and (ii) dashed lines 64 and 74 of graphs 60 and 70, respectively, are indicative of first and second events of change in medication administered to patient B. respectively.

In some embodiments, the rise up of graph 70 in a section 76 (before dashed line 74), and in sections 78 and 79b (before dashed line 75) predicts the need for the first and second hospitalization events of patient B, respectively. As described above, a simultaneous steep rise rate of both the RVL and the mean LAP is indicative of a rapid CHE in the heart. Moreover, an example of a strong correlation between the mean LAP and the RVL calculated in daily measurements within sections 79a and 79b, is depicted below.

In some embodiments, (i) the calculated mean LAP and RVL values of sections 68a and 68b, respectively, could be used as a base line for monitoring the heart condition of patient B. (ii) dashed line 73 is indicative of a reduction in the medication administered to patient B (relative to the original prescription administered during the first hospitalization (marked by dashed line 72), (iii) the sharp RVL rise rate (of about 4 mmHg/day) in section 76 results from the medication reduction described above, and (iv) at the second medication change marked by dashed line 74, the cardiologist changed the medication from the reduced level back to the original level described above.

In general, some of the sections such as section 68b of graph 70, have relatively slow rate of change, so that in some embodiments, the processor is configured to identify such sections, and to use the calculated parameters at such sections as a base line. Moreover, the processor is configured to identify and use sections having a fast rate of change in the calculated parameters (such as sections 76, 78, 79a and 79b), as a prediction tool to assist medical treatment.

In some embodiments, the processor is configured to hold one or more zones, which are indicative of the cardiac condition of the patient. For example, a “green zone” refers to a range of mean LAP values that are indicative of optimal cardiac conditions of the patient. As such, the cardiac condition of the patient does not require hospitalization or a change in medication. The zones may be predefined for a group of patients (e.g., based on statistical analysis of a plurality of patients having similar cardiac conditions), or for a single patient (e.g., based on conditions fit to be used as a baseline). Additionally, or alternatively, the zone may be determined by the rise rate and/or fall rate of a selected parameter, such as mean LAP, RVL or any other suitable parameter. Moreover, a “yellow zone” refers to a deterioration in the cardiac conditions, and a “red zone” refers to a rapidly deteriorating and/or severe cardiac condition. In the example of graphs 60 and 70, the LAP, mean Lap, and RVL values of section 68 (and the corresponding section in graph 60), may be used as a base line to a green zone, the calculated rise rate of section 76 or section 78 may be used as a baseline for the yellow zone, which requires a change of medication but not necessarily hospitalization. Moreover, the calculated rise rate of sections 79a and 79b as well as the calculated rise rates of sections 61 and 71 as a whole, may be used as red zones that require both immediate hospitalization and changes in medication. It is noted that the predefined zones may be altered between patients, and may be determined for any suitable set of one or more of the parameters described herein.

In some embodiments, based on a sharp rise rate in section 78, the second hospitalization event of patient B could take place earlier than the time indicated by dashed line 75. For example, if the second hospitalization would have been carried out between sections 78 and 79a of graph 70, then the sharp rise rate (e.g., of about 4.1 mmHg/day) in both sections 79a and 79b, which is indicative of an exacerbation in the heart condition, could be avoided or at least reduced.

In some embodiments, a sharp rise rate in section 76 of graph 70 could provide the cardiologist with an early warning, so that the medication change could take place earlier than the time indicated by dashed line 74.

In some embodiments, based on the fall rate in the RVL after the second medication change (e.g., between dashed line 74 and section 78) the processor is configured to provide the cardiologist with the response of the patient to the medication change and/or to any other treatment administered to the patient.

In the example of FIG. 4A, based on the estimated parameters, such as the relative V-LAP (RVL) of graph 70 and the mean LAP of graph 60 described above, embodiments of the present invention can be used for predicting the occurrence of a cardiac condition, such as CHF. in a patient suffering from a heart failure exacerbation and being monitored using system 11. More specifically, using system 11 and the disclosed techniques, the cardiologist can advance (i) the altering of the drug administration to the patient and/or (ii) the hospitalization of the patient.

Moreover, it is noted that in the example of FIGS. 3A and 3B, the correlation between the graphs of the mean LAP and the RVL is weak, so that graphs 51 and 57 of the RVL provide the cardiologist with the medical condition of the patient's heart. In the example of FIG. 4A, both graph 60 of the mean LAP, and graph 70 of the RVL, provide the cardiologist with a prediction or early warning of CHF being developed. Moreover, (i) as will be depicted in FIG. 4C below, a strong correlation between sections 79a and 79b of graphs 60 and 70, respectively, is indicative of a rapid deterioration in the cardiac condition and congestion, and because the quality of LAP measurement is sufficiently high (ii) a weak correlation between sections 68a and 68b of graphs 60 and 70, respectively, is indicative of stability in the cardiac condition and normal fluid volume of patient B, as will be depicted in FIG. 4B below.

It is noted that embodiments of the present disclosure use high resolution LAP data, e.g., data of one or more components (e.g., AW, VW, and RP) collected within of a heartbeat cycle of the heart, rather than collecting and/or averaging LAP of one or more entire heartbeat cycles. For example, the embodiments described in FIGS. 3B, 3B, and 4A may be applied, mutatis mutandis, to the LAP data related to the Apeak (e.g., Apeak 43 shown in FIG. 2 above).

It is noted, however, that in some cases the AW and Apeak may not be presented on the respective graph, e.g., due to a clinical state (i.e., the cardiac condition) of the heart of the respective patient. In such cases, the embodiments related to the AW and the Apeak LAP data are not applicable.

In some embodiments, the processor is configured to predict a heart failure exacerbation based on the RVL as a standalone parameter. For example, the processor is configured to predict heart failure deterioration: (i) when identifying an absolute increase in the level of RVL above a predefined value (e.g., determined by the zones described above, and/or by the RVL level between section 76 and dashed line 74, and/or (ii) when identifying the aforementioned rise rate of 3.37 mmHg/day in section 61.

As described above, LAP is considered to be the standard physiological indicator of congestion. The disclosed techniques use the RVL and other parameters (e.g., RAL) described IN FIG. 2 above for more accurate estimation of the congestion status of the patient, and predicting occurrence of heart failure deterioration. Based on the prediction, the cardiologist determines a proactive treatment (e.g., using diuretics) in order to reduce or prevent the congestion. In some cases, the quality and accuracy of the LAP measurement may be reduced due to: (i) noise caused by a technical source, such as the drift in implant 24 described in FIGS. 3A and 3B above, and/or (ii) noise caused by a physiological source, such as the respiration affecting the level of pressure in the patient chest and heart. Both origins of noise interfere with the LAP measurements.

In some embodiments, the RVL and RAL parameters are used by the processor for cancelling both sources of noise, because both RVL and RAL, are calculated by subtracting the mean LAP of the LAP measurement, from the LAP measurements at the peak of the V wave and the A wave, respectively. As such, RVL and RAL are immune to the aforementioned noise, and can be used for assessing and predicting the congestion status of the heart more accurately compared to LAP measurements and/or calculated mean LAP.

Additionally, or alternatively, the processor is configured to predict a heart failure exacerbation based on the level of correlation between the RVL and the mean LAP, as depicted in detail in FIGS. 4B and 4C below.

FIG. 4B is a schematic presentation of a graph 101 depicting a level of correlation between the mean LAP and RVL calculated based on LAP measurements acquired in sections 68a and 68b of FIG. 4A, respectively, in accordance with embodiments of the present invention.

In some embodiments, the processor is configured to calculate (i) mean LAP, and (ii) average RVL, for each WF produced within the time intervals of sections 68a and 68b, respectively. As described in the example of FIG. 2 above, each WF is produced based on approximately 1500 LAP measurements acquired by implant 24 within approximately 15 seconds. The LAP measurements may be carried out on a daily basis, or using any other suitable monitoring frequency.

In some embodiments, the processor is configured to calculate a pearson correlation between the average RVL and the mean LAP of each WF, which are presented in graph 101. In the present example, the calculated value of pearson correlation is weak, approximately 0.46, which is indicative of a stable cardiac condition and fluid volume status (e.g., no congestion) of patient B. In other words, in a stable cardiac condition, the variability in the volume of fluid is low, so that any noise in the measurements reduces the correlation.

In other embodiments, instead of calculating the pearson correlation, the processor is configured to calculate any other suitable correlation between the average RVL and the mean LAP of each WF. Additionally, or alternatively, the processor is configured to calculate any suitable correlation between an average of the RAL and the mean LAP of each WF.

In some embodiments, the processor is configured to set one or more predefined thresholds for the level of one or more of the correlations described above, respectively. As such, when the value of a given calculated correlation exceeds the respective predefined threshold, the processor is configured to output a prediction of a HFE (e.g., a severe level of CHF).

FIG. 4C is schematic presentation of a graph 103 depicting a level of correlation between the mean LAP and RVL calculated based on LAP measurements acquired in sections 79a and 79b of FIG. 4A, respectively, in accordance with embodiments of the present invention.

In some embodiments, the processor is configured to calculate (i) mean LAP, and (ii) average RVL, for each WF produced within the time intervals of sections 79a and 79b, respectively. Each WF is produced based on approximately 1500 LAP measurements, as described in FIGS. 2 and 4B above.

In some embodiments, the processor is configured to calculate the pearson correlation between the average RVL and the mean LAP of each WF, which are presented in graph 103. In the present example, the calculated value of pearson correlation is very strong, approximately 0.99. Based on the one or more thresholds described above, the pearson correlation of about 0.99 exceeds the threshold, which is indicative of a rapid deterioration and a severe level of congestion and CHF exacerbation. It is note that the strong correlation is indicative of the severe level of CHF, because every increase in the level of LAP immediately increases the level of Vpeak, and therefore, also the calculated level of both the mean LAP and the RVL. In other words, in a severe level of congestion and CHF exacerbation, the compliance of the left atrium is being reduced exponentially and when the compliance is low, the V peak, LAP and RVL all increase rapidly together, and therefore, the correlation is high in the example of FIG. 4C. Such rapid deterioration requires hospitalization of patient B, and administering an aggressive treatment using diuretics to reduce the level of congestion.

### Analyzing the Correlation Between Mean Lap and the Peak of V Wave

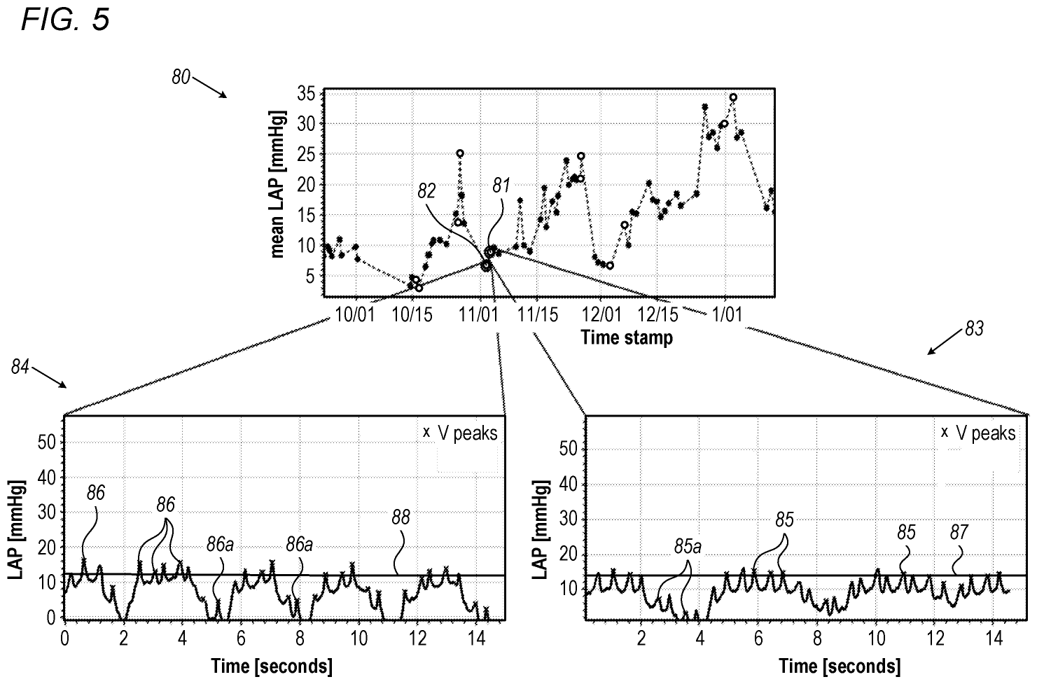

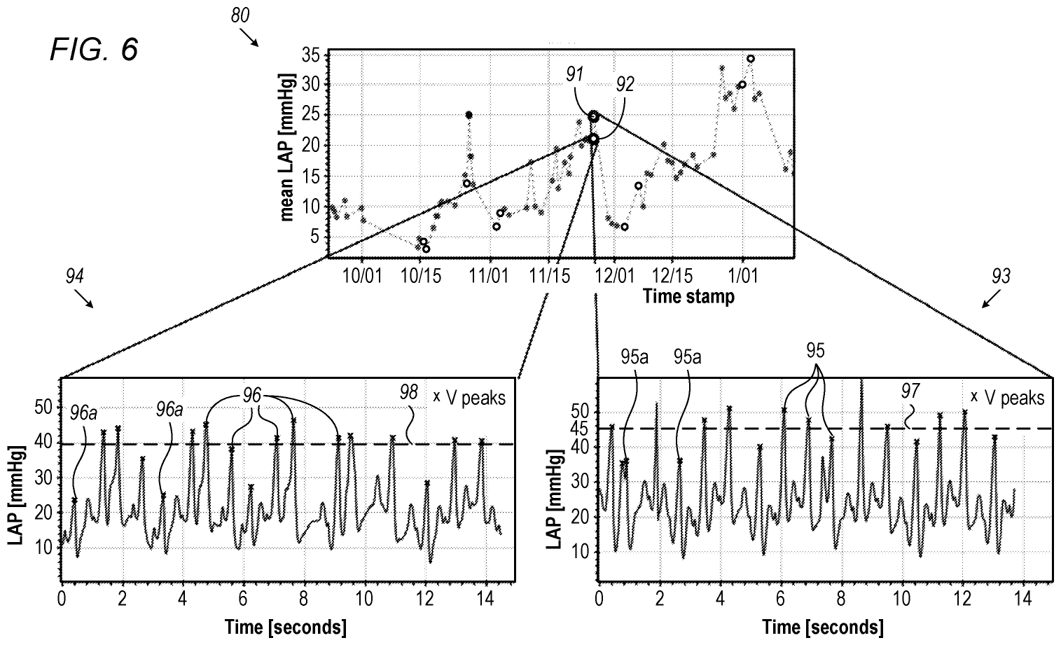

FIGS. 5 and 6 are schematic presentations of points 81 and 82, and points 91 and 92. respectively, which are annotated over a graph 80 depicting the mean LAP calculated based on LAP measurements carried out over about 7.5 months in patient B using system 11, in accordance with embodiments of the present invention.

Note that graph 80 is based on the same LAP measurements as graph 60 of FIG. 4A above, but in the example of graph 80, the 5-day moving average was not applied to the LAP measurements.

Reference is now made to FIG. 5. In some embodiments, the processor is configured to present over graph 80, points 81 and 82 that are annotating relatively low values of mean LAP measurements.

Reference is now made to graphs 83 and 84 presenting the mean LAP acquired at points 81 and 82, respectively, during a time interval of about 15 seconds each. In some embodiments, the processor is configured to annotate Vpeaks 85 and Vpeaks 86 in graphs 83 and 84, respectively. Note that the processor also annotates in graphs 83 and 84, Vpeaks 85a and Vpeaks 86a, respectively. The LAP values of Vpeaks 85a and Vpeaks 86a are lower than that of Vpeaks 85 and Vpeaks 86, respectively, due to the respiration cycle of patient B.

In some embodiments, the processor is configured to apply a high-pass filter (e.g., larger than about 0.3 Hz and 0.4 Hz) to the measurement data, so as to remove low frequency respiration effects on the measured pressure waveform.

In some embodiments, the processor is configured to calculate average values 87 and 88 of Vpeaks 85 and Vpeaks 86, respectively. In the present example, the mean values at points 81 and 82 are about 8 mmHg and 7 mmHg, respectively. Moreover, the average values 87 and 88 of points 81 and 82, are about 12 mmHg and 11 mmHg, respectively. In other words, the mean LAP and the average Vpeak are in correlation, and both remain relatively low.

Reference is now made to FIG. 6. In some embodiments, the processor is configured to present over graph 80, points 91 and 22 that are annotating relatively high values of mean LAP measurements.

Reference is now made to graphs 93 and 94 presenting the mean LAP acquired at points 91 and 92, respectively, during a time interval of about 15 seconds each (similar to that of graphs 83 and 93 of FIG. 5 above). In some embodiments, the processor is configured to annotate Vpeaks 95 and Vpeaks 96 in graphs 93 and 94, respectively. Note that the processor also annotates in graphs 93 and 94, Vpeaks 95a and Vpeaks 96a, respectively. The LAP values of Vpeaks 95a and Vpeaks 96a are lower than that of Vpeaks 95 and Vpeaks 96, respectively, due to the respiration cycle of patient B (as also described in FIG. 5 above).

Moreover, as described in FIG. 5 above, the processor is configured to apply a high-pass filter (e.g., larger than about 0.3 Hz or 0.4 Hz) to the measurement data, so as to remove low frequency respiration effects on the measured pressure waveform.

In some embodiments, the processor is configured to calculate average values 97 and 98 of Vpeaks 95 and Vpeaks 96, respectively. In the present example, the mean values at points 91 and 92 are about 25 mmHg and 20 mmHg, respectively. Moreover, the average values 97 and 98 of points 81 and 82, are about 45 mmHg and 40 mmHg, respectively. In other words, the mean LAP and the average Vpeak are in correlation, and both remain relatively high.

In some embodiments, the strong correlation between the mean LAP and the calculated average of the Vpeak, both in high values and low values of LAP, are indicative that at least in the example of patient B, both mean LAP and the RVL (which is the difference between the mean LAP and the calculated average of the Vpeak at every point in graph 80) can be used for predicting heart failure exacerbation (e.g., CHF) and/or response to treatment. Moreover, the inventors found that approximately the same correlation (between the mean LAP and the RVL) is also maintained in a plurality of patients regardless of the level of LAP (i.e., high, medium, and low levels of LAP).

In some embodiments, the rise rate in the RVL (and/or in any other suitable parameter, such as the mean LAP described above, and the RAL described in FIG. 2 above), can be used to predict the need for (i) hospitalization of patients (e.g., patient B), and/or (ii) change of medication administered to these patients. Moreover, in the example of FIGS. 4A-6, the fall rate in at least the RVL (and potentially in one or more of the other parameters described in FIG. 2 above) can be indicative of the effect of the medication on the cardiac condition of the patients consuming them.

In other embodiments, the prediction based on the rise rate and fall rate could be different (e.g., inverse) to that described in FIGS. 4A-6 above. For example, two parameters that are based on the WF analysis could have an inverse correlation during a CHF event.

### Personalizing Management of Congestive Heart Failure Among Different Patients

FIG. 7A is a schematic presentation of a graph 5 depicting parameters calculated based on LAP measured in the hearts of patients C, D, and E (not shown) other than patient 30, using system 11, in accordance with an embodiment of the present invention.

In some embodiments, the processor is configured to plot in graph 5 the calculated RVL parameter (i.e., the value of mean LAP subtracted from the values of Vpeak) as a function of the calculated mean LAP parameter, both parameters are calculated based on the LAP measured by implant 24 in patients C, D, and E.

In some embodiments, the processor is configured to determine three sections in graph 5, e.g., sections 7, 8 and 9 defined by vertical dashed lines 6. It is noted that the relationship between (i) the RVL (which is the mean LAP subtracted from the Vpeak), and (ii) the mean LAP, is altered in the different sections among at least one of these three different patients.

For example, with reference to patients D and E, in section 7, the ratio between the RVL and mean LAP appears to be approximately similar. In sections 8 and 9, the slope gets steeper in the example of patient E compared to that of patient D, so that in sections 8 and 9 the ratio between the RVL and mean LAP appears to be substantially different between patient D and E. Moreover, the slope of the RVL/mean LAP ratio of patient E is steeper compared to that of patient C as well. As such, the mean LAP parameter is not sufficient for reliable predicting the occurrence of acute CHF exacerbation in a patient.

In some embodiments, based on the RVL data of graph 5, the processor is configured to predict rapid exacerbation in patient E, which requires immediate intervention by the healthcare staff, while the cardiac condition of patients C and D appears to be stable.

In such embodiments, based on the RVL, and/or the ratio between the RVL and mean LAP, the processor is configured to: (i) identify differences in the biomechanical cardiac properties between different patients, and thereby, (ii) enable a personalized set of monitoring thresholds and treatment for each individual patient.

FIG. 7B is a schematic presentation of a graph 111 depicting parameters calculated based on LAP measured in the hearts of patients F, G, and H (not shown, other than patients 30, C, D, and E), using system 11, in accordance with an embodiment of the present invention.

In some embodiments, the processor is configured to plot in graph 111 the calculated RVL parameter (i.e., the value of mean LAP subtracted from the values of Vpeak) as a function of the mean LAP parameter, both parameters are calculated based on the LAP measured by implant 24 in patients F, G, and H.

Dashed lines 113 and 114 refer to the actual hospitalization date of patients H and G. respectively.

In some embodiments, the waveform analysis provides users of system 11 (e.g., the cardiologist) with insights to the biomechanical condition and comorbidities of the heart of each individual patient. Such insights are not available while relying solely on LAP measurements, and calculated mean LAP.

In the present example, in section 115 defined between dashed lines 113 and 114, the calculated values of mean LAP of patients F, G, and H, are similar, e.g., between about 19 mmHg and 31 mmHg, but the cardiac conditions of patients F, G, and H, are completely different from one another.

In some embodiments, the calculated values of the RVL, and the ratio between the RVL and the mean LAP, provide the cardiologist with the aforementioned insights to the cardiac condition of each individual patient. In the example of patient H, a calculated RVL value of about 12 mmHg, is indicative of HFE that requires hospitalization, while the value of the calculated mean LAP is relatively low, e.g., about 19 mmHg.

In the example of patient G, at a calculated mean LAP value of about 19, the calculated RVL value is about 8 mmHg, and the cardiac condition of patient G is stable. As shown in graph 111, patient G had been hospitalized with a calculated mean LAP value of about 31, and a calculated RVL value between about 14 mmHg and 16 mmHg. Moreover, in the example of patient F. (i) hospitalization was not required even when the calculated value of the mean LAP exceeded about 30 mmHg, and (ii) at mean LAP levels between about 23 mmHg and 31 mmHg, the correlation between RVL and mean LAP appears to be weak, which is also indicative that the cardiac condition of patient F is stable, as described in detail in FIGS. 4A and 4B above.

As such, it is clear that the calculated mean LAP cannot serve as a reliable monitor to predict HFE in different patients.

In some embodiments, based on the hospitalization events of patient G and F and the calculated parameters of graph 111, and in order to predict HFE, the processor is configured to hold an RVL threshold: (i) smaller than about 12 mmHg for patient H, and (ii) smaller than about 14 mmHg for patient G.

In such embodiments, the waveform analysis, and calculated parameters such as RVL, enable personalization of the monitoring zones and type of treatment administered to each individual patient. Moreover, based on the example of graph 111, mean LAP could not be used for monitoring a plurality of patients, each having a different biomechanical structure and functionality of the heart.

Moreover, graph 111 shows that for each individual patient, a strong correlation between RVL and mean LAP is indicative of HFE (as described in detail in FIG. 4C above), but the LAP baseline and the ratio between RVL and mean LAP could be individual to each patient. In other words, the WF analysis and the calculated parameters, such as RVL, are indicative of different susceptibility to deterioration of the cardiac condition among different patients.

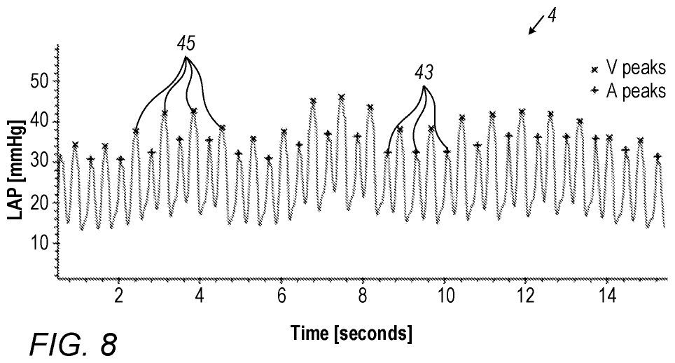

FIG. 8 is a schematic presentation of a graph 4 depicting techniques for estimating Apeaks 43 and Vpeaks 45 based on LAP measurements acquired over about 15 seconds using system 11, in accordance with an embodiment of the present invention.

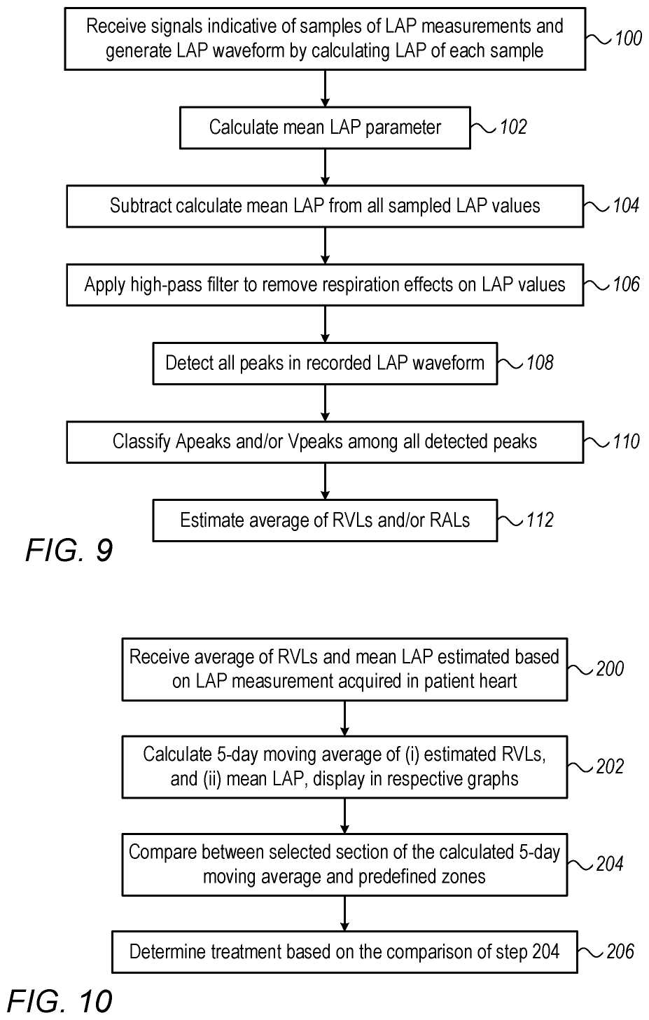

In some embodiments, the processor receives signals indicative of LAP measurements from implant 24, the LAP measurement sampling is carried out during at least one heartbeat cycle, for example, during about 15 heartbeat cycles (having a total duration of about 15 seconds as described above). The processor (of cloud 15 and/or processor 22) is configured to run software comprising an algorithm configured to perform calculations based on a method depicted in FIG. 9 below.

FIG. 9 is a flow chart that schematically illustrates a method for estimating Vpeaks 45, average RVL, Apeaks 43, and average RAL, in accordance with an embodiment of the present invention.

The method begins at a waveform generation step 100, with the processor receiving from implant 24 signals indicative of samples of LAP measurements, and generating one or more LAP waveforms (such as the WF shown for example in FIG. 2 above) by calculating the LAP value of each sample.

At a mean LAP calculation step 102, the processor calculates the mean LAP parameter by averaging the values of all the samples of the LAP over the entire sampling period, for example, 15 seconds as described in detail in FIGS. 2, 5-6, and 8 above.

At a mean LAP subtraction step 104, the processor subtracts the value of the mean LAP (calculated in step 102 above) from all the sampled LAP values.

At a respiration effect filtering step 106, the processor applies a high-pass filter (e.g., larger than about 0.3 Hz or 0.4 Hz) to remove the low frequency respiration effects on the measured pressure waveform (as described, for example, in FIGS. 5 and 6 above).

At a peak detection step 108, the processor detects all the peaks in the LAP waveform generated in step 100 above, as shown for example in FIGS. 5, 6 and 7 above.

At a peak classification step 110, the processor classifies the peaks to the Apeaks 43 and/or Vpeaks 45 shown in graph 4. In some embodiments, the classification is carried out using properties of the detected peaks extracted from sampled values of the LAP at the vicinity of each peak. In some embodiments, the processor is configured to use the following properties of the WF to perform the classification:
Maximal LAP value.
Width of the peak (measured in seconds) at various LAP values.
Rise rate defined as (i) the difference between the maximal LAP value and minimal LAP value at the beginning of the wave, (ii) divided by the time difference between the points having the maximal and minimal LAP values.
Fall rate defined as (i) the difference between the maximal LAP value and the minimal LAP value at the end of the wave, (ii) divided by the time difference between the points having the maximal and minimal LAP values.

At an RVL and RAL estimation step 112 that concludes the method, the processor estimates:
(i) the average RVL, which is the average of the maximal LAP value in all of the detected Vpeaks after subtraction of the mean LAP, and/or
(ii) the average RAL, which is the average of the maximal LAP value in all of the detected Apeaks after subtraction of the mean LAP.

FIG. 10 is a flow chart that schematically illustrates a method for treating CHF in the patient heart based on the RVL calculated in FIG. 9 above, in accordance with an embodiment of the present invention.

The method begins at an RVL receiving step 200, with the processor receiving (or extracting from the memory of cloud 15 and/or computer 12) the RVL values estimated based on (i) the signals indicative of LAP measurements received from implant 24, and (ii) applying, to the LAP measurements, the method described in FIG. 9 above. Moreover, the processor may receive (or extract from the memory) also the mean LAP described above.

It is noted that the LAP measurements are performed (using implant 24) in one or more sessions every day (e.g., typical duration of each session is at least 15 seconds), and the processor produces WFs and graphs, such as graph 40 and WFs described in detail in FIG. 2 above. Moreover, the monitoring of the patient's heart is carried out over days or weeks or months, as described in detail in FIG. 4A above.

At a moving average calculation step 202, the processor calculates a 5-day moving average of: (i) the RVLs estimated in step 200 above, and (ii) the mean LAP. Moreover, the processor is configured to present the calculated 5-day moving averages of the RVL and the mean LAP in a graph, such as the graphs shown and described in detail in FIGS. 3A, 3B and 4 above.

At a comparison step 204, the processor compares between (i) the calculated 5-day moving average of the RVL and mean LAP of a selected section of interest in the graph, and (ii) predefined values of the zones described in FIG. 4A above. Additionally, or alternatively, and as described in the example of FIG. 4A above, the processor is configured to compare the values of the mean LAP and RVL of the selected section with (i) section 68 whose RVL values are indicative of a base line, and (ii) one or more RVL and mean LAP values of section 76 (in close proximity to dashed line 74), and sections 79a and 79b, whose RVL and mean LAP values are indicative of development of CHF.

Moreover, as shown described in graphs 60 and 70 of FIG. 4A above, the processor is configured to compare between (i) a calculated trend of the (a) RVL, and (b) mean LAP of the selected section of interest, and (ii) the rise rate calculated in (a) section 61, and (b) section 71, respectively.

At a treatment determination step 206 that concludes the method, based on the one or more comparison(s) of step 204 above, the processor is configured to provide the cardiologist with a recommended treatment based on treatment protocols of CHF and other sorts of heart failure exacerbations.

In some embodiments, after the cardiologist determines the treatment, and the medication is administered to the patient, the processor is configured to estimate the impact of the treatment on the cardiac condition of the patient. As described in the example of FIG. 4A above, the fall rate of graphs 60 and 70 between dashed line 74 and sections 61 and 71. respectively, can be used for assessing the effectiveness of the drug administered to patient B in after the second medication change marked by dashed line 74.

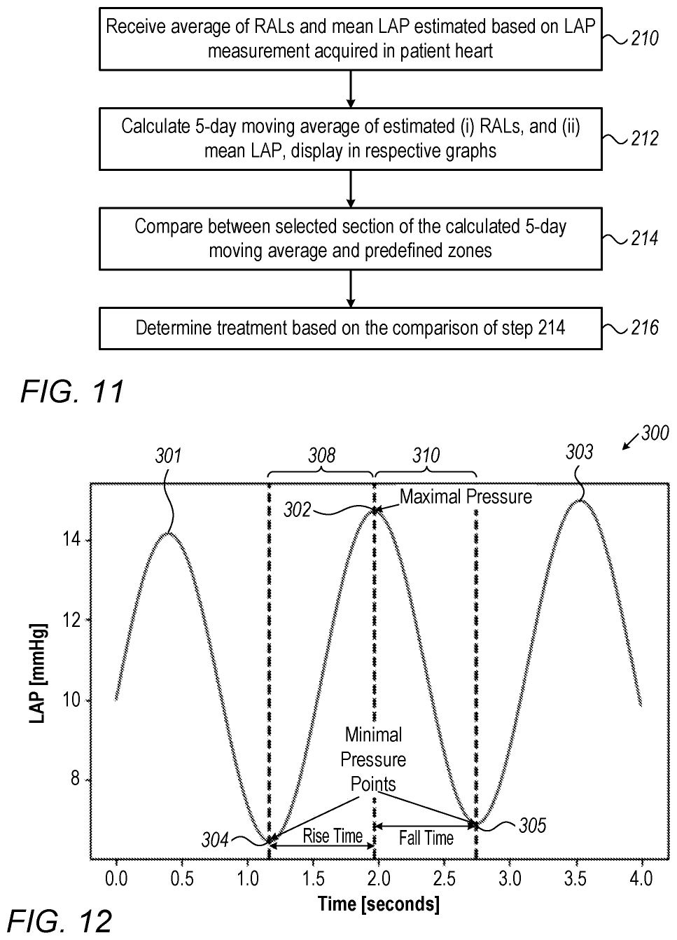

FIG. 11 is a flow chart that schematically illustrates a method for treating CHF in the patient heart based on the RAL calculated in FIG. 9 above, in accordance with an embodiment of the present invention.

The method begins at an RAL receiving step 210, with the processor receiving (or extracting from the memory of cloud 15 and/or computer 12) the RAL values estimated based on (i) the signals indicative of LAP measurements received from implant 24, and (ii) applying, to the LAP measurements, the method described in FIG. 9 above. Moreover, the processor may receive (or extract from the memory) also the mean LAP described above.

It is noted that the LAP measurements are performed (using implant 24) in one or more sessions every day (e.g., typical duration of each session is at least 15 seconds), and the processor produces WFs and graphs, such as graph 40 and WFs described in detail in FIG. 2 above. Moreover, the monitoring of the patient's heart is carried out over days or weeks or months, as described in FIG. 4A above.

At a moving average calculation step 212, the processor calculates a 5-day moving average of: (i) the RALs estimated in step 210 above, and (ii) the mean LAP. Moreover, the processor is configured to present the calculated 5-day moving averages of the RAL and the mean LAP in a graph, as described in FIG. 10 above.

At a comparison step 214, the processor compares between (i) the calculated 5-day moving average of the RAL and mean LAP of a selected section of interest in the graph, and (ii) predefined values of the zones described in FIG. 4A above, and or to values of selected sections of the graph, such as section 68b of graph 70 depicted in FIG. 4A above. Step 214 is applied using the same techniques, mutatis mutandis, applied to calculated RVL, as described for the RVL-based treatment in FIG. 10 above.

Additionally, or alternatively, based on the same techniques described for the RVL in graphs 60 and 70 of FIG. 4A above, the processor is configured to compare between (i) a calculated trend of the (a) RAL, and (b) mean LAP of the selected section of interest, and (ii) the rise rate calculated in respective sections of the graph. In the example of FIG. 4A the rise rate of the RVL is calculated in (a) section 61, and (b) section 71, respectively.

At a treatment determination step 216 that concludes the method, based on the one or more comparison(s) of step 214 above, the processor is configured to provide the cardiologist with a recommended treatment based on treatment protocols of CHF and other sorts of heart failure exacerbations.

In some embodiments, after the cardiologist determines the treatment, and the medication is administered to the patient, the processor is configured to estimate the impact of the treatment on the cardiac condition of the patient, using the same techniques, mutatis mutandis, described in FIG. 10 above.

FIG. 12 is a schematic presentation of a graph 300 of simulated signals indicative of a WF of LAP measurements, and calculation of several parameters based on the simulated WF, in accordance with an embodiment of the present invention.

Embodiments related to graph 300 depict techniques for detecting peaks in the LAP measurements of graph 300, and for estimating several parameters, such as but not limited to (i) peak to peak delta LAP (P2P), (ii) LAP amplitude, (iii) rise rate and/or rise time of the LAP measurements, and (iv) fall rate and/or fall time of the LAP measurements.

In some embodiments, the processor (of cloud 15 and/or processor 22) receives signals indicative of LAP measurements from implant 24, the LAP measurement sampling is carried out during at least one heartbeat cycle, in the present example, during about 4 seconds).

In some embodiments, the processor is configured to run software comprising one or more algorithm configured to detect all the peaks in the graph, in the present example, peaks 301, 302, 303, 304, and 305 of graph 300.

In some embodiments, the processor is further configured to classify peaks 301-305 to (i) peak of maximum LAP value also referred to herein as max peak 301, max peak 302, and max peak 303, and (ii) peak of minimum LAP value also referred to herein as min peak 304. and min peak 305. In the present example, the max peaks comprise Vpeaks, but the techniques described below are also applicable, mutatis mutandis, to Apeaks, and/or to any other suitable peaks detected in a graph of LAP measurements. It is noted that the terms “maximum,” and “max,” and the terms “minimum.” and “min” refer to local maximum and local minimum of the respective WF. In other words, each WF may have different values of maximum and minimum LAPs, compared to that of the other WFs of the same graph. As such, the peaks are detected based on the maximum and minimum values of the LAP within each WF.

Moreover, it is noted that the measured LAP values may vary (i) among the max peaks, and (ii) among the min peaks, the variations may be caused by (a) natural variations in the level of contraction and relaxation of the heart muscle(s), and (b) the respiration wave having a typical frequency of about 0.2 Hz, as described in more detail above.

In some embodiments, based on the max peaks and min peaks, the processor is configured to estimate rise rates, rise times, fall rates, and fall times in the graphs. In the present example, the rise rate and rise time are estimated in a section 308 defined between min peak 304 and max peak 302. Similarly, the fall rate and fall time are estimated in a section 310 defined between max peak 302 and min peak 305. In other words, the rise rates and rise times are calculated between a min peak and a subsequent adjacent max peak, and the fall rates and fall times are calculated between a max peak and a subsequent adjacent min peak, as shown in graph 300 and described above.

In other embodiments, the processor is configured to calculate the rise rate and/or rise time in section 308 of graph 300 using: (i) a LAP value of about 10% larger than that of peak 304 (instead of using the LAP value of peak 304), and (ii) a LAP value of about 10% smaller than that of peak 302 (instead of using the LAP value of peak 302). Similarly, the processor is configured to calculate the fall rate and/or fall time in section 310 by using: (i) a LAP value of about 10% smaller than that of peak 302 (instead of using the LAP value of peak 302), and (ii) a LAP value of about 10% larger than that of peak 305 (instead of using the LAP value of peak 305). In other words, instead of using the max peak and min peak values, the processor can perform the rise rate and/or time and fall rate and/or time calculations, using about 90% and about 10% of the graph amplitude, respectively. It is noted that this technique is applicable, mutatis mutandis, to all the Vpeaks, Apeaks, and other sorts of peaks that appear and depicted in all the embodiments and the Figs. of the present disclosure.

In some embodiments, the processor is configured to estimate the rise time and the fall time parameters based on the horizontal axis of graph 300. For example, the rise time between peaks 304 and 302 is about 0.8 seconds, and the fall time between peaks 302 and 305 is about 0.75 seconds.

In some embodiments, the processor is configured to estimate the rise rate and the fall rate parameters based on: (i) peak-to-peak delta LAP (P2P) shown on the vertical axis of graph 300, and (ii) the estimated rise time and fall time described above. For example, the P2P between peaks 302 and 304 is about 8 mmHg, and the P2P between peaks 302 and 305 is about 7.7 mmHg. The processor is configured to estimate the rise rate parameter between peaks 304 and 302 by dividing 8 mmHg (of the P2P) by 0.8 seconds (of the rise time), so that the estimated rise rate equals about 10 mmHg/second. Similarly, the processor is configured to estimate the fall rate parameter between peaks 302 and 305 by dividing 7.7 mmHg (of the P2P) by 0.75 seconds (of the fall time), so that the estimated rise rate equals about 10.27 mmHg/second.

It is noted that the estimated values of the above parameters may alter by being calculated using other pairs of max and min peaks. For example, when applying the above estimations and calculations between peaks 301 and 304, and between peaks 305 and 303, the values of one or more of the parameters estimated above may be altered compared to the corresponding estimated values described above.

In some embodiments, the processor is configured to estimate the level of a LAP amplitude, e.g., by calculating the mean LAP of all the present measurements (such as the mean LAP 46 of FIG. 2 above), and calculating the delta LAP by subtracting the mean LAP from a selected peak. It is noted that an absolute value is applied to the subtraction output when estimating the LAP amplitude between a min peak and the mean LAP.

In some cases, the monitoring of the patient's heart LAP is carried out over days or weeks or months, and at least one of the estimated parameters may be altered in response to changes in the cardiac conditions, as described for example in FIG. 4A above.

In some embodiments, based on the LAP measurements received from implant 24, the processor is configured to detect and calculate changes over time, in the estimated values of one or more of the parameters described above. In one implementation, variations in the estimated values of the Vpeak and RVL (i.e., Vpeak-mean LAP) are described in detail in FIG. 4A above. In another example, the estimated variations in the rise rate and/or fall rate of the Vpeak may be calculated using linear regression between the estimated values of the Vpeak over time, and the time and date of each measurement. In this example, the processor is configured to extract a first coefficient that indicates the rise rate and/or fall rate of the change in the LAP value of the Vpeak per day or per any other selected time interval. Similarly, the processor is configured to apply the same technique to the mean LAP, and to extract a second coefficient that indicates the rise rate and/or fall rate of the mean LAP per day or per any other selected time interval.

In other embodiments, the processor is configured to perform the estimations described above using any suitable techniques other than the linear regression described above.

In some embodiments, the processor is configured to store (e.g., in a memory of cloud 15 and/or computer 12) previous values of at least one of the first and second coefficients, which have been extracted from the same patient in the past, for example, several weeks ago. The processor is configured to compare between the values of each pair of corresponding present and previous coefficients, so as to determine whether or not the currently estimated rate of change over time are indicative of a real prediction of heart failure exacerbation. In one implementation of this technique, the processor is configured to calculate a moving average of the data, such as the 5-day moving average or using any other suitable time interval to carry out the moving average calculation. As described above, the same techniques are applicable, mutatis mutandis, for estimating the rate of change over time of any of the other parameters described above. For example, the time interval used for calculating the moving average may be altered based on the level of fluctuations in the data of the estimated parameter collected over time, e.g., more fluctuations require a longer time interval for smoothing the data.

In some embodiments, the processor is configured to estimate the heart rate (i.e., the rate of heartbeats) and/or respiration rate (i.e., the rate of respiration) of patient 30 (and any of the other patients described above) based on: (i) the pressure measurements received from implant 24, and (ii) the estimated peaks (e.g., Vpeak and/or Apeak), and calculated mean LAP described in detail above.

In some embodiments, the processor is configured to estimate the heart rate by: (i) applying a low-pass filter (e.g., smaller than about 50 Hz) to the pressure measurements, so as to remove high-frequency noise effects on the WF of the pressure measurements, (ii) calculating a normalized periodogram of the waveform, which shows the power of the waveform at different frequencies, and (iii) identify, in the periodogram, the frequency having the maximum power at a predetermined range of frequencies (e.g., between about 0.5 Hz and 3 Hz). As such, the identified frequency is the estimated frequency of the heart rate. The term periodogram refers to an estimation of the spectral density of the waveform, which may be implemented using any suitable known technique, such as but not limited to Fast Fourier Transform (FFT) spectrum analysis. An output of such spectral analysis comprises a graph (not shown) having the frequency of the spectrum in the horizontal axis of the graph, and the power (also referred to herein as intensity) of each of the frequencies on the vertical axis of the graph. Thus, the frequency having the highest power among all the frequencies of the graph, is identified as the estimated frequency of the heart rate.

Additionally, or alternatively to the above embodiments, the processor is configured to estimate the respiration rate by: (i) applying a low-pass filter (e.g., smaller than about 50 Hz or about 25 Hz) to the pressure measurements, so as to remove high-frequency noise effects on the WF of the pressure measurements, (ii) calculating a normalized periodogram of the waveform, which shows the power of the waveform at different frequencies, and (iii) identify, in the periodogram, the frequency having the maximum power at a predetermined range of frequencies (e.g., between about 0.1 Hz and 0.5 Hz or any other suitable range depending on the condition of the respective patient). As such, the identified frequency is the estimated frequency of the respiration rate.

In other embodiments, the processor is configured to estimate the heart rate by: (i) applying a high-pass filter (e.g., larger than about 0.3 Hz or 0.4 Hz) to the pressure measurements, so as to remove low-frequency respiration effects on the measured pressure waveform, subsequently (ii) detecting the peaks remaining after applying the aforementioned high-pass filter, and classifying the peaks to Apeaks and Vpeaks, using at least part of the technique described in FIG. 9 above. In such embodiments, the processor is configured to estimate the heart rate of the patient by: (a) counting the number of Apeaks and/or Vpeaks, and (b) dividing the number of Apeaks and/or Vpeaks, by the duration of the time interval in which the WFs were produced (based on the pressure measurements received from implant 24), as described in detail, for example, in one or both of FIGS. 2 and 8 above.

It is noted that in some cases the AW (A wave) and Apeak may not be presented on the respective graph, e.g., due to a clinical state (i.e., the cardiac condition) of the heart of the respective patient. In such cases, the embodiments related to the AW and the Apeak LAP data are not applicable.

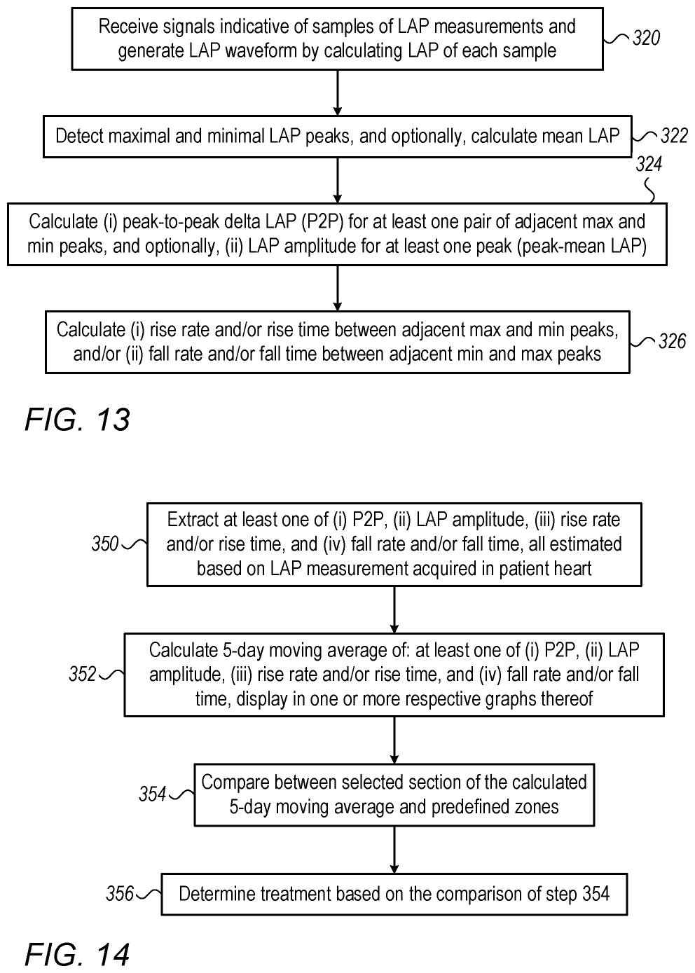

FIG. 13 is a flow chart that schematically illustrates a method for estimating (i) rise rate and/or rise time, and (ii) fall rate and/or fall time between adjacent maximum and minimum peaks of LAP measurements, in accordance with an embodiment of the present invention.

The method begins at a waveform generation step 320, with the processor receiving from implant 24 signals indicative of samples of LAP measurements, and generating one or more LAP waveforms (such as the WF shown for example in FIG. 2 above) by calculating the LAP value of each sample.

At a peak detection step 322, the processor detects peaks indicative of local maximum and minimum LAP values, as described in detail, for example in FIG. 12 above. Optionally, the processor may also calculate the mean LAP of the entire graph, as described for example in FIG. 2 above.

At a first parameter calculation step 324, the processor calculates at least one of the: (i) peak-to-peak delta LAP (P2P) for at least one pair of adjacent max and min peaks, and optionally, (ii) LAP amplitude for at least one peak (peak-mean LAP), as described in detail, for example in FIG. 12 above.

At a second parameter calculation step 326 that concludes the method, the processor calculates the: (i) rise rate and/or rise time between adjacent max and min peaks, and/or (ii) fall rate and/or fall time between adjacent min and max peaks, as described in detail, for example in FIG. 12 above. In some embodiments, all the parameters calculated in steps 324 and 326 are being stored in the memory of cloud 15, and optionally, also in the memory of computer 12.

Alternatively, instead of using the values of the maximum and minimum peaks, the processor can perform the rise rate and/or time and fall rate and/or time calculations of the method of FIG. 13 using about 90% and about 10% of the graph amplitude, respectively, as described in FIG. 12 above.

FIG. 14 is a flow chart that schematically illustrates a method for treating CHF in the patient heart based on at least one of the (i) P2P. (ii) LAP amplitude, (iii) rise rate and/or rise time, and (iv) fall rate and/or fall time that are calculated in FIG. 13 above, in accordance with an embodiment of the present invention.

The method begins at a parameter extracting step 350, with the processor extracting from the memory of cloud 15 and/or the memory of computer 12, at least one of the (i) P2P. (ii) LAP amplitude, (iii) rise rate and/or rise time, and (iv) fall rate and/or fall time parameters, which are all estimated based on LAP measurement acquired in patient heart, as described in detail, for example in FIGS. 12 and 13 above.

At a moving average calculation step 352, the processor calculates a 5-day moving average of at least one of the parameters extracted in step 350 above. More specifically, the parameters are: (i) P2P, (ii) LAP amplitude, (iii) rise rate and/or rise time, and (iv) fall rate and/or fall time. Moreover, the processor is configured to present the calculated 5-day moving averages of one or more of the aforementioned parameters in one or more respective graphs, as described for example in FIGS. 10 and 11 above.

At a comparison step 354, the processor compares between (i) the calculated 5-day moving average of one of the selected parameters of step 352 above, which is calculated for a selected section of interest in the respective graph, and (ii) predefined values of the zones described in FIG. 4A above, and or to values of selected sections of the graph, such as section 68b of graph 70 depicted in FIG. 4A above. Step 354 is applied using the same techniques, mutatis mutandis, applied to any of the (i) P2P, (ii) LAP amplitude, (iii) rise rate and/or rise time, and (iv) fall rate and/or fall time parameters described above. Moreover, the comparison technique is described in detail, but for other parameters, in FIG. 10 above.

At a treatment determination step 356 that concludes the method, based on the one or more comparison(s) of step 354 above, the processor is configured to provide the cardiologist with a recommended treatment based on treatment protocols of CHF and other sorts of heart failure exacerbations.

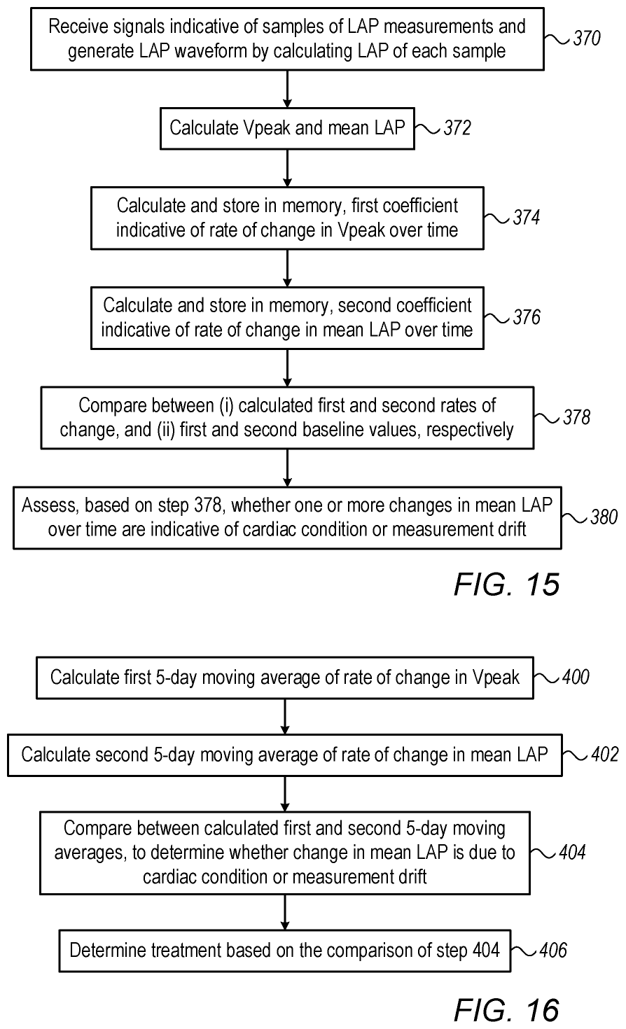

FIG. 15 is a flow chart that schematically illustrates a method for estimating change rate in Vpeak and mean LAP, and for assessing whether the mean LAP change rate is applicable for treating CHF, in accordance with an embodiment of the present invention.

The method begins at a waveform generation step 370, with the processor receiving from implant 24 signals indicative of samples of LAP measurements, and generating one or more LAP waveforms (such as the WF shown for example in FIG. 2 above) by calculating the LAP value of each sample.

At a first calculation step 372, the processor calculates the Vpeak and mean LAP in the waveform(s) generated in step 370 above, as described for example in FIG. 2 above.

At a second calculation step 374, the processor calculates and stores in the memory (of cloud 15 and/or computer 12) a first coefficient indicative of the rate of change in Vpeak over time, as described in detail, for example in FIG. 4A above.

In some embodiments, in order to extract the coefficient that indicates the rate of change in Vpeak per day, the processor calculates the rate of change in V peak using linear regression between (i) Vpeak values over time and (ii) respective time and date of each measurement.

At a third calculation step 376, the processor calculates and stores in the memory (of cloud 15 and/or computer 12) a second coefficient indicative of the rate of change in mean LAP over time, as described in detail, for example in FIG. 4A above. In some embodiments, in order to extract the coefficient that indicates the rate of change in mean LAP per day, the processor calculates the rate of change in mean LAP using any suitable technique, such as but not limited to linear regression, between (i) mean LAP values over time and (ii) respective time and date of each measurement. For example, instead of linear regression, the processor is configured to use one or more techniques selected from a list of techniques consisting of: Moving average rate of change, Exponential moving average rate of change, Applying a smoothing spline and calculating the rate of change of the interpolated spline curve, Applying Autoregressive Integrated Moving Average (ARIMA) or SARIMA which includes seasonality differencing and using the “d” or differencing parameter to assess the rate of change, Applying filtering techniques such as low-pass filters and calculating the rate of change on the filtered output, Using Time window analysis, also known as moving or “rolling” window analysis with various window durations to calculate the rate of change. It is noted that such techniques may be used, instead of or in addition to linear regression, at all the embodiments and methods of the present disclosure that describe the use of linear regression.

At a comparison step 378, the processor compares between the (i) calculated first and second rates of change, and (ii) first and second baseline values, respectively, which are (i) calculated when the conditions of the patient fit for baseline calculation, and/or (ii) predefined values of the zones described in FIG. 4A above. For example, with reference to FIG. 4A above, the baseline of the RVL (which is the mean LAP subtracted from the Vpeak), is calculated based on section 68b of graph 70.

At an assessment step 380 that concludes the method, based on the comparison of step 378 above, the processor is configured to assess whether one or more changes in mean LAP over time are indicative of cardiac condition or drift caused by the measurement carried out by implant 24. In the examples of FIGS. 3A and 3B, the fall rate and rise rate shown in sections 52 and 58, respectively, are assessed and identified by the processor as false positives for CHF being developed in the hearts of the respective patients, as described in detail in FIGS. 3A and 3B above. In the example of FIG. 4A, the rise rate shown in both sections 61 and 71 of graphs 60 and 70, respectively, are assessed and identified by the processor as true positive, which is indicative of the CHF being developed in patient B, as described in detail in FIG. 4A above.

FIG. 16 is a flow chart that schematically illustrates a method for treating CHF in the patient heart based on the calculated change rate in at least one of the Vpeak and the mean LAP described in FIG. 15 above, in accordance with an embodiment of the present invention.

The method begins at a first moving average calculation step 400, with the processor calculating a first 5-day moving average of the rate of change (e.g., rise rate and/or fall rate) in Vpeak, as described in detail, for example in FIGS. 4A and 15 above.

At a second moving average calculation step 402, the processor calculates a second 5-day moving average of the rate of change (e.g., rise rate and/or fall rate) in mean LAP, as described in detail, for example in FIGS. 4A and 15 above.

At a comparison step 404, the processor is configured to compare between the calculated first and second 5-day moving averages of steps 400 and 402, respectively. Based on the comparison, the processor is configured to determine whether change of rate in mean LAP is: (i) due to cardiac condition, as described in the example of FIG. 4A above, or (ii) due to measurement drifts, as described in the examples of FIGS. 3A and 3B above.

In some embodiments, the processor is further configured to compare between (i) one or both of the calculated first and second 5-day moving averages of steps 400 and 402, and (ii) predefined values of the zones described in FIG. 4A above.

At a treatment determination step 406 that concludes the method, based on the comparison of step 404 above, the processor is configured to provide the cardiologist with a recommended treatment based on treatment protocols of CHF and other sorts of heart failure exacerbations.

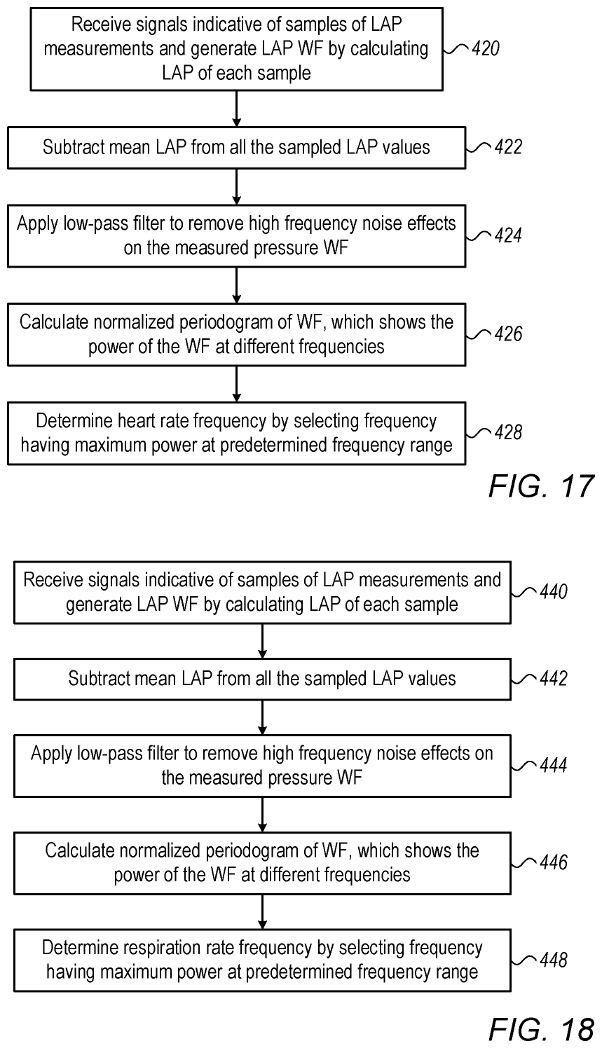

FIG. 17 is a flow chart that schematically illustrates a method for estimating the heart rate of patient 30 (and the other patients described above) based on the LAP measurements received from implant 24, in accordance with an embodiment of the present invention.

The method begins at a waveform generation step 420, with the processor receiving from implant 24 signals indicative of samples of LAP measurements, and generating one or more LAP waveforms (such as the WF shown for example in FIG. 2 above) by calculating the LAP value of each sample.

At a first calculation step 422, the processor calculates the mean LAP in the waveform(s) generated in step 420 above, as described for example in FIG. 2 above. Moreover, the processor subtracts the mean LAP from all the sampled LAP values, as described in detail in FIG. 2 above.

At a filtering step 424, the processor applies a low-pass filter (e.g., smaller than about 50 Hz) to the calculated output of step 422 above, as described in detail in FIG. 12 above.

At a second calculation step 426, the processor calculates a normalized periodogram of the one or more WFs, and generates the power of the one or more WFs at different frequencies, as described in detail in FIG. 12 above.

At a heart rate determination step 428 that concludes the method, based on the output of step 426 above, the processor is configured to determine the frequency of the heart rate. In some embodiments, the processor is configured to select the frequency having maximum power (among the frequencies shown in the output of step 426) within a predetermined range of frequencies, e.g., between about 0.5 Hz and 3 Hz, as described in detail in FIG. 12 above.

FIG. 18 is a flow chart that schematically illustrates a method for estimating the respiration rate of patient 30 (and the other patients described above) based on the LAP measurements received from implant 24, in accordance with an embodiment of the present invention.

The method begins at a waveform generation step 440, with the processor receiving from implant 24 signals indicative of samples of LAP measurements, and generating one or more LAP waveforms (such as the WF shown for example in FIG. 2 above) by calculating the LAP value of each sample.

At a first calculation step 442, the processor calculates the mean LAP in the waveform(s) generated in step 440 above, as described for example in FIG. 2 above. Moreover, the processor subtracts the mean LAP from all the sampled LAP values, as described in detail in FIG. 2 above.

At a filtering step 444, the processor applies a low-pass filter (e.g., smaller than about 50 Hz) to the calculated output of step 442 above, as described in detail in FIG. 12 above.

At a second calculation step 446, the processor calculates a normalized periodogram of the one or more WFs, and generates the power of the one or more WFs at different frequencies, as described in detail in FIG. 12 above.

At a respiration rate determination step 448 that concludes the method, based on the output of step 446 above, the processor is configured to determine the frequency of the respiration rate. In some embodiments, the processor is configured to select the frequency having maximum power (among the frequencies shown in the output of step 446) at a predetermined range of frequencies, e.g., between about 0.1 Hz and 0.5 Hz or any other suitable range depending on the condition of the respective patient, as described in detail in FIG. 12 above.

In some embodiments, the methods of FIGS. 17 and 18 may be applied to the Vpeaks and/or to the Apeaks of the corresponding one or more WFs. It is noted, however, that in some cases the AW and Apeak may not be presented or distinguishable on the respective graph, e.g., due to a cardiac condition of the heart of the respective patient. In such cases, the embodiments related to the AW and the Apeak LAP data are not applicable.

Although the embodiments described herein mainly address managing chronic heart failure based on left atrial pressure measurements, the methods and systems described herein can also be used in other applications, such as in measurements of right atrial pressure (RAP), and pulmonary capillary wedge pressure (PCWP), which are measured using any suitable pressure sensor. Moreover, the disclosed techniques could be used, mutatis mutandis, in pressure measurement applications in pulmonary arteries and/or pulmonary ventricles. Furthermore, the disclosed techniques could be used, mutatis mutandis, in measurements other than pressure, for example, in electrocardiogram (ECG) and other suitable types of measurements acquired in organs of a patient.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method, comprising:
receiving a plurality of measurements of blood pressure acquired in a heart of a patient;
deriving, from the measurements, a periodic waveform of the blood pressure, and estimating one or more parameters of one or more components of the periodic waveform, respectively; and
predicting occurrence of a cardiac condition in the patient based on the estimated one or more parameters.

2. The method according to claim 1, wherein receiving the measurements comprise receiving multiple ones of the measurements of the blood pressure per cardiac cycle.

3. The method according to claim 1, wherein the blood pressure measurements comprise Left Atrial Pressure (LAP) measurements acquired by a cardiac implant.

4. The method according to claim 1, wherein the one or more components of the periodic waveform comprise at least one of: (i) a ventricle wave (VW) generated in response to a passive filling of an atrium of the heart with oxygenated blood, and (ii) an atrial wave (AW) generated in response to an active contraction of the atrium.

5. The method according to claim 4, wherein estimating the one or more parameters comprises estimating at least one of: (i) a first peak pressure of the VW (Vpeak), and (ii) a second peak pressure of the AW (Apeak).

6. The method according to claim 5, wherein the blood pressure measurements comprise Left Atrial Pressure (LAP), and wherein estimating the one or more parameters comprise calculating a mean LAP, which is an average of the measurements of the LAP in the periodic waveform.

7. The method according to claim 6, and comprising detecting in the periodic waveform: (i) a first local minimum LAP at a first side of the Vpeak, and (ii) a second local minimum LAP at a second side of the Vpeak, opposite the first side.

8. The method according to claim 7, wherein estimating the one or more parameters comprise estimating a rise rate, by (i) calculating a first LAP difference between the first local minimum LAP and the Vpeak, and (ii) dividing the first LAP difference by a rise time parameter, which is a first time interval between the first local minimum LAP and the Vpeak.

9. The method according to claim 7, wherein estimating the one or more parameters comprise estimating a fall rate, by (i) calculating a second LAP difference between the Vpeak and the second local minimum LAP, and (ii) dividing the second LAP difference by a fall time parameter, which is a second time interval between the Vpeak and the second local minimum LAP.

10. The method according to claim 6, wherein estimating the one or more parameters comprise estimating at least one of: (i) a relative ventricle LAP (RVL), by subtracting the mean LAP from the Vpeak, and (ii) a relative atrial LAP (RAL), by subtracting the mean LAP from the Apeak.

11. The method according to claim 10, wherein when (i) the mean LAP exhibits a trend as a function of time and (ii) at least one of the RVL and RAL does not exhibit the trend, predicting occurrence of the cardiac condition is based on at least one of the RVL and RAL.

12. The method according to claim 11, and comprising calibrating the acquisition of the measurements of the blood pressure responsively to a difference between the mean LAP and at least one of RVL and RAL.

13. The method according to claim 10, wherein when both (i) the mean LAP and (ii) at least one of the RVL and RAL exhibit a trend as a function of time, predicting occurrence of the cardiac condition is based on the trend of one or both of: (a) the mean LAP, and (b) at least one of the RVL and RAL.

14. The method according claim 10, and comprising plotting: (i) a first graph of a first moving average of the mean LAP as the function of time, and (ii) one or more second graphs of second moving averages of one or both of the RVL and the RAL as the function of time, respectively.

15. The method according to claim 10, and comprising, calculating a correlation between the mean LAP and at least one of RVL and RAL, and determining a threshold indicative of an occurrence of a heart failure exacerbation (HFE), and wherein predicting occurrence of the cardiac condition comprises predicting the HFE when the calculated correlation exceeds the threshold.

16. The method according to claim 1, wherein the measurements exhibit a trend as a function of time, and wherein estimating the parameter comprises canceling at least part of the trend.

17. The method according to claim 1, wherein estimating the one or more parameters comprises: (a) identifying in the periodic waveform: (i) one or more first peaks indicative of one or more maximum values of the blood pressure within one or more time intervals of the periodic waveform, respectively, (ii) one or more second peaks indicative of one or more minimum values of the blood pressure within the one or more time intervals, respectively, and (b) estimating a pressure difference between each pair of the first and second peaks within each of the time intervals.

18. The method according to claim 17, and comprising predicting the occurrence of the cardiac condition based on the one or more estimated pressure differences.

19. The method according to claim 17, wherein estimating the one or more parameters comprises: (a) calculating a mean blood pressure, which is an average of the measurements of the blood pressure in the periodic waveform, and (b) estimating a pressure amplitude by subtracting the mean blood pressure from at least one of the first and second peaks, and comprising predicting the occurrence of the cardiac condition based on the estimated pressure amplitude.

20. The method according to claim 1, and comprising determining, for at least a given parameter among the one or more parameters, at least a first range of first values and a second range of second values different from the first values, and wherein predicting the occurrence of the cardiac condition comprises comparing between: (a) a given value of the given parameter, and (b) the first and second ranges of the first and second values.

21. The method according to claim 20, and comprising determining at least one of: (i) a first treatment to the patient, in case the given value is within the first range, (ii) a second treatment to the patient, in case the given value is within the second range, and (iii) a third treatment to the patient, in case the given value is out of the first and second ranges.

22. The method according to claim 1, and comprising (i) receiving a plurality of additional measurements of another blood pressure acquired in another heart of an additional patient; (ii) deriving, from the additional measurements, an additional periodic waveform of the another blood pressure, and estimating the one or more parameters of the one or more components identified in the additional periodic waveform, respectively; and (iii) predicting the occurrence of the cardiac condition in the additional patient based on the estimated one or more parameters.

23. The method according to claim 22, and comprising setting (i) a first threshold for predicting the occurrence of the cardiac condition in the patient, and (ii) a second threshold, different from the first threshold, for predicting the occurrence of the cardiac condition in the additional patient.

24. A system, comprising:
an interface, which is configured to receive a plurality of measurements of blood pressure acquired in a heart of a patient; and
a processor, which is configured to:
derive, from the measurements, a periodic waveform of the blood pressure, and estimate one or more parameters of one or more components of the periodic waveform, respectively; and
predict occurrence of a cardiac condition in the patient based on the estimated one or more parameters.

25. The system according to claim 24, wherein the interface is configured to receive multiple ones of the measurements of the blood pressure per cardiac cycle.

26. The system according to claim 24, wherein the blood pressure measurements comprise Left Atrial Pressure (LAP) measurements acquired by a cardiac implant.

27. The system according to claim 24, wherein the one or more components of the periodic waveform comprise at least one of: (i) a ventricle wave (VW) generated in response to a passive filling of an atrium of the heart with oxygenated blood, and (ii) an atrial wave (AW) generated in response to an active contraction of the atrium.

28. The system according to claim 27, wherein the processor is configured to estimate the one or more parameters by estimating at least one of: (i) a first peak pressure of the VW (Vpeak), and (ii) a second peak pressure of the AW (Apeak).

29. The system according to claim 28, wherein the blood pressure measurements comprise Left Atrial Pressure (LAP), and wherein the processor is configured to estimate the one or more parameters by calculating a mean LAP, which is an average of the measurements of the LAP in the periodic waveform.

30. The system according to claim 29, wherein the processor is configured to detect in the periodic waveform: (i) a first local minimum LAP at a first side of the Vpeak, and (ii) a second local minimum LAP at a second side of the Vpeak, opposite the first side.

31. The system according to claim 30, wherein the processor is configured to estimate the one or more parameters by estimating a rise rate, by (i) calculating a first LAP difference between the first local minimum LAP and the Vpeak, and (ii) dividing the first LAP difference by a rise time parameter, which is a first time interval between the first local minimum LAP and the Vpeak.

32. The system according to claim 30, wherein the processor is configured to estimate the one or more parameters by estimating a fall rate, by (i) calculating a second LAP difference between the Vpeak and the second local minimum LAP, and (ii) dividing the second LAP difference by a fall time parameter, which is a second time interval between the Vpeak and the second local minimum LAP.

33. The system according to claim 29, wherein the processor is configured to estimate the one or more parameters by estimating at least one of: (i) a relative ventricle LAP (RVL), by subtracting the mean LAP from the Vpeak, and (ii) a relative atrial LAP (RAL), by subtracting the mean LAP from the Apeak.

34. The system according to claim 33, wherein when (i) the mean LAP exhibits a trend as a function of time and (ii) at least one of the RVL and RAL does not exhibit the trend, the processor is configured to predict occurrence of the cardiac condition based on at least one of the RVL and RAL.

35. The system according to claim 34, wherein, responsively to a difference between the mean LAP and at least one of RVL and RAL, the processor is configured to recommend calibration the of acquisition of the measurements of the blood pressure.

36. The system according to claim 33, wherein when both (i) the mean LAP and (ii) at least one of the RVL and RAL exhibit a trend as a function of time, the processor is configured to predict occurrence of the cardiac condition based on the trend of one or both of: (a) the mean LAP, and (b) at least one of the RVL and RAL.

37. The system according to claim 33, wherein the processor is configured to plot: (i) a first graph of a first moving average of the mean LAP as the function of time, and (ii) one or more second graphs of second moving averages of one or both of the RVL and the RAL as the function of time, respectively.

38. The system according to claim 33, wherein the processor is configured to: (i) calculate a correlation between the mean LAP and at least one of RVL and RAL, (ii) determine a threshold indicative of an occurrence of a heart failure exacerbation (HFE), and (iii) predict the HFE when the calculated correlation exceeds the threshold.

39. The system according to claim 24, wherein the measurements exhibit a trend as a function of time, and wherein the processor is configured to estimate the parameter by canceling at least part of the trend.

40. The system according to claim 24, wherein the processor is configured to estimate the one or more parameters by: (a) identifying in the periodic waveform: (i) one or more first peaks indicative of one or more maximum values of the blood pressure within one or more time intervals of the periodic waveform, respectively, (ii) one or more second peaks indicative of one or more minimum values of the blood pressure within one the or more time intervals, respectively, and (b) estimating a pressure difference between each pair of the first and second peaks within each of the time intervals.

41. The system according to claim 40, the processor is configured to predict the occurrence of the cardiac condition based on the one or more estimated pressure differences.

42. The system according to claim 40, wherein the processor is configured to estimate the one or more parameters by: (a) calculating a mean blood pressure, which is an average of the measurements of the blood pressure in the periodic waveform, and (b) estimating a pressure amplitude by subtracting the mean blood pressure from at least one of the first and second peaks, and wherein the processor is configured to predict the occurrence of the cardiac condition based on the estimated pressure amplitude.

43. The system according to claim 28, wherein the processor is configured to: (i) determine, for at least a given parameter among the one or more parameters, at least a first range of first values and a second range of second values different from the first values, and (ii) predict the occurrence of the cardiac condition by comparing between (a) a given value of the given parameter, and (b) the first and second ranges of the first and second values.

44. The system according to claim 43, wherein the processor is configured to determine at least one of: (i) a first treatment to the patient, in case the given value is within the first range, (ii) a second treatment to the patient, in case the given value is within the second range, and (iii) a third treatment to the patient, in case the given value is out of the first and second ranges.

45. The system according to claim 24, wherein the interface is configured to receive a plurality of additional measurements of another blood pressure acquired in another heart of an additional patient; and wherein the processor is configured to: (i) derive, from the additional measurements, an additional periodic waveform of the another blood pressure, and estimate the one or more parameters of the one or more components identified in the additional periodic waveform, respectively; and (ii) predict the occurrence of the cardiac condition in the additional patient based on the estimated one or more parameters.

46. The system according to claim 45, wherein the processor is configured to set (i) a first threshold for predicting the occurrence of the cardiac condition in the patient, and (ii) a second threshold, different from the first threshold, for predicting the occurrence of the cardiac condition in the additional patient.
